(19) United States
(12) Patent Application Publication
(10) Pub. No.: US 2015/0119251 A1
(43) Pub. Date: Apr. 30, 2015

(54) AGRICULTURAL PLANT-PROTECTING AGENTS CONTAINING DIPEPTIDE DERIVATIVE AS ACTIVE INGREDIENT

(71) Applicants: REPUBLIC OF KOREA (RURAL DEVELOPMENT ADMINISTRATION), Suwon, Gyeonggi-do (KR); NAPRO BIOTEC INC., Gongju-si, Chungcheongnam-do (KR)

(72) Inventors: Kyung Seok Park, Suwon-si (KR); Jin Woo Park, Suwon-si (KR); Se Won Lee, Suwon-si (KR); Surk Sik Moon, Gongju-si (KR); In Seok Hong, Daejeon (KR)

(21) Appl. No.: 14/395,422

(22) PCT Filed: Apr. 18, 2013
(86) PCT No.: PCT/KR2013/003303

§ 371 Date: Jan. 15, 2015

(30) Foreign Application Priority Data
Apr. 18, 2012 (KR) 10-2012-0040279

(51) Int. Cl. A01N 43/50; A01N 37/18; C07K 5/065; A01N 43/38; A01N 41/12; C07K 5/062; C07K 5/078

(52) U.S. Cl. CPC A01N 43/50; C07K 5/06026; A01N 37/18; C07K 5/06147; C07K 5/06156; A01N 43/38; A01N 41/12; C07K 5/06078
USPC 504/275; 560/169; 504/319; 562/561; 504/320; 548/338.1; 548/507; 504/284; 560/37

(57) ABSTRACT

Provided is an agricultural plant-protecting agent including a dipeptide derivative or an agro-pharmaceutically acceptable salt thereof as an active ingredient, which has a plant disease-preventing effect, a plant growth-promoting effect, and a plant immunity-activating effect.

### TECHNICAL FIELD

The present invention relates to an agricultural plant-protecting agent including a dipeptide derivative or an agro-pharmaceutically acceptable salt thereof as an active ingredient, which has a plant disease-preventing effect, a plant growth-promoting effect, and a plant immunity-activating effect.

### BACKGROUND ART

The plants make signaling molecules in order to defend themselves when they are invaded by pathogens or physically wounded. It is known that such signaling molecules has the effects as plant defense activators, plant strengthening agents, or plant immunity activators. As the representative signaling molecules of the plants, salicylic acid and jasmonic acid have been known. When the defense reaction of plant is operated due to outside invasions, salicylic acid, a signaling molecule, induces the genes, such as, PR-1, BGL-2, PR-5, SID-2, EDS-5, and PAD-4 to be expressed in the plant, and jasmonic acid induces the genes, such as, PDF1.2, VSP, HEL, THI-2, FAD3, ERS1, and ERF1 to be expressed in the plant [Dong, X., Current Opinion in Plant Biology, 1998, 1, 316-323; Glazebrook, J., Current Opinion in Plant Biology, 1999, 2, 280-286; Bostock, R. M., Physiology and Molecular Plant Pathology, 1999, 55, 99-109]. Particularly, the PR-1 gene is a gene indicating the resistance-inducing phenomenon of a plant, which is generated by salicylic acid, the expression of the PR-1 gene is a crucial sign of operation of the signaling process by a plant. When a PR protein is expressed in a plant, the plant shows a disease resistant effect such as an antibacterial activity. In addition, a PDF1.2 gene is a gene indicating the resistance-inducing phenomenon of a plant, which is induced by Jasmonic acid, the expression of the PDF1.2 gene is a crucial sign of the progress of the signaling process for operation of the defense reaction of plant.

Benzo-1,2,3-thiadiazole-7-carbothioic acid S-ethyl ester (BTH) has been commercially used as a signaling molecule of a plant. BTH is a synthetic compound having the similar chemical structure as salicylic acid. It is reported that BTH shows an activity of suppressing Blumeria graminis that causes a barley powdery mildew, the ozone resistance of a plant, the resistance to Botrytis cinerea in a grape plant, a promoting activity of the biosynthesis of resveratrol and anthocyanin, the resistance to a powdery mildew in a strawberry, and also an accumulating effect of phenolic compounds. BTH has a function of inducing the expression of the PR-1 gene like salicylic acid does, but does not induce the expression of PDF1.2 gene exhibited by jasmonic acid.

As described above, when the signaling molecules such as salicylic acid and jasmonic acid for the self-defense of a plant induce to express the genes such as PR-1 or PDF1.2, the plant can suppress the propagation of the pathogens and also can endure a physical wound. In other words, it is considered that self-defense ability is induced in a plant. Therefore, even if the conventional antimicrobial agent or germicide is not treated, when such genes are expressed in a plant, the plant can resist the pathogen. Therefore, it is considered that the molecule expressing such genes has a great value as an agricultural drug for a plant.

Meanwhile, a dipeptide is a peptide molecule having an amide bond generated by condensing two amino acids. The dipeptide is generated in various types of structures even at the time of performing the proteolysis, and also is generated even during the process of synthesizing 2,5-diketopiperazine, a cyclic dipeptide. Aspartame, that is, an artificial sweetener, belongs to a dipeptide being composed of two amino acids. As being reported so far, a dipeptide compound is mainly applied as an artificial sweetener, a platelet aggregation inhibitor, and the like [International Patent Publication No. WO9616981, U.S. Pat. Nos. 5,968,581 and 5,763,408, and Europe Patent Publication No. 513675].

The present inventors synthesized the dipeptide derivatives prepared by two amino acids, and found that when these compounds are treated to a plant, they express the plant disease-resisting genes such as PR-1, glucanase, chitinase, PR4, peroxidase, and PR10. In addition, we found that when the dipeptide derivatives are treated to a plant before forming the lesion, the formation of the lesion in a plant is significantly suppressed, and also in addition to the plant disease-preventing effect, a plant growth-promoting effect and a plant immunity-increasing effect (in detail, a plant disease-resisting ability or plant cooling-preventing ability) are also improved. As a result, the present inventors completed the present invention.

In other words, the present inventors found at first time that the dipeptide derivatives induce the disease resistance gene to be expressed, and shows a plant disease-preventing effect by suppressing the proliferation of pathogen, a plant growth-promoting effect, and a plant immunity-increasing, and up to now, no case has been reported in any literature.

### DISCLOSURE

### Technical Problem

An object of the present invention is to provide an use of a dipeptide derivative as an agricultural plant-protecting agent.

### Technical Solution

In order to achieve such an object, the present invention provides an agricultural plant-protecting agent including a compound selected from a dipeptide derivative represented by the following chemical formula 1 or an agro-pharmaceutically acceptable salt thereof as an active ingredient.

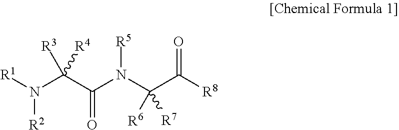

In the above chemical formula 1,

R¹, R², and R⁵ are the same or different from each other, and represent a hydrogen atom, a linear or branched C₁-C₁₈ alkyl carbonyl group, a linear or branched C₁-C₁₈ alkoxy carbonyl group,

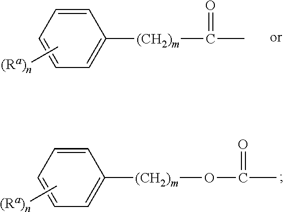

R³, R⁴, R⁶, and R⁷ are the same or different from each other, and represent a hydrogen atom, or a linear or branched C₁-C₁₈ alkyl group substituted or unsubstituted with the group selected from hydroxy, mercapto, amino, guanidino, N,N-bis(benzyloxycarbonyl)guanidino, carbamoyl, carboxylic acid, linear or branched C₁-C₁₈ alkoxy carbonyl, linear or branched C₁-C₁₈ alkenyl oxycarbonyl,

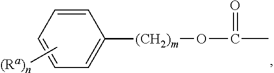

linear or branched C₁-C₁₈ alkyl thio, trityl thio, acetyl amino, phenyl, hydroxyphenyl, imidazole, and indolyl;

or any one of the R³ and R⁴ may bind to R² to form a nitrogen-containing 5-membered to 7-membered ring or any one of the R⁶ and R⁷ may bind to R⁵ to form a nitrogen-containing 5-membered to 7-membered ring.

R⁸ represents hydroxy; a linear or branched C₁-C₁₈ alkoxy group; a linear or branched C₁-C₁₈ alkyl amino group;

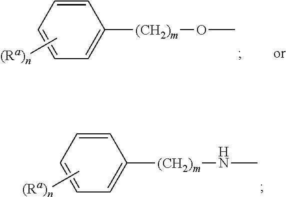

R^{a} represents a linear or branched C₁-C₁₈ alkyl group;

n represents an integer of 0 to 5, the number of a substituent, R^{a}; and

m represents an integer of 0 to 6.

In addition, the present invention provides a method for preventing or suppressing a plant disease, in which the method includes treating an agricultural plant-protecting agent including a compound selected from a dipeptide derivative represented by the above chemical formula 1 or an agro-pharmaceutically acceptable salt thereof as an active ingredient to a plant.

In addition, the present invention provides a method for activating plant immunity, in which the method includes treating an agricultural plant-protecting agent including a compound selected from a dipeptide derivative represented by the above chemical formula 1 or an agro-pharmaceutically acceptable salt thereof as an active ingredient to a plant.

In addition, the present invention provides a method for promoting plant growth, in which the method includes treating an agricultural plant-protecting agent including a compound selected from a dipeptide derivative represented by the above chemical formula 1 or an agro-pharmaceutically acceptable salt thereof as an active ingredient to a plant.

### Advantageous Effects

The agricultural plant-protecting agent of the present invention has an effect on significantly reducing the formation of lesion in a plant due to the suppressions of infection and proliferation of pathogens by inducing the production of the plant disease-resisting protein by expressing the plant disease-resisting genes such as PR-1, glucanase, chitinase, PR4, peroxidase, and PR10, when being treated to a plant. In addition, the agricultural plant-protecting agent of the present invention allows a plant to activate the disease resistant mechanism to pathogen or protect itself from chilling damages by operating self-defense mechanism. In addition, the agricultural plant-protecting agent of the present invention allows a plant to tolerate environmental stress caused by cold or hot weather condition, and also has an effect on promoting plant growth.

In addition, the agricultural plant-protecting agent of the present invention has a systemic effect on other regions which was not treated, by the self-defense mechanism of a plant. In other words, even though the agent is treated to a part of a plant, such as seeds, roots, stems, and leaves of a plant, it has an effect on other untreated regions, and even on harvested fruits.

Since the agricultural plant-protecting agent has an excellent effect on preventing plant diseases such as bacterial rot, damping-off, phytophthora blight, a wilt disease, a leaf spot disease or a mosaic disease, which are caused by a bacteria, a virus, or a mold, it is useful as an environment-friendly agricultural plant-protecting agent capable of replacing the conventional germicides and being used.

The agricultural plant-protecting agent of the present invention has an excellent effect in the case of applying to dicotyledoneae, for example, solanaceae such as potatoes, peppers, sweet peppers, and tomatoes, cucurbitaceae, such as cucumbers, cigarettes, watermelons, and oriental melons, a crucifer (cruciferae) such as Chinese cabbage, lettuces, radishes, cabbages, rapes, peanuts, and a salary, a medicinal plant, such as ginseng and dong quai, a plant, such as perilla seeds, strawberry, spring onion, garlic, ginger, and onion, graminae, such as, rice, barley, corn, and sorghum, and fruit trees, such as apple tree, pear tree, peach tree, and persimmon tree.

### DESCRIPTION OF DRAWINGS

FIG. 1 is a diagram illustrating the degrees of the expressions of the defense genes expressed in the cases of treating Compound 80 and Compound 89 to a pepper plant. In FIG. 1, (a) and (b) are the results of the gene expressions which were measured in 12 and 24 hours after inoculating Pectobacterium carotovora causing a bacterial rot after 1 week later since Compound 80 was treated to a pepper seedling. In FIG. 1, (c) and (d) are the results showing the degrees of the defense genes expressions generated after 12 and 24 hours since Compound 80 was treated and phytophthora (Phytophthora capcisi) was inoculated (1. Control group; 2. Pathogen treatment; 3. Treatment of 0.1 mM BTH; 4. Treatment of 0.1 mM BTH and pathogen; 5. Treatment of 0.1 ppm of Compound 80; 6. Treatment of 0.1 ppm of Compound 80 and pathogen; 7. Treatment of 1.0 ppm of Compound 80; 8. Treatment of 1.0 ppm of Compound 80 and pathogen; 9. Treatment of 10.0 ppm of Compound 80; 10. Treatment of 10.0 ppm of Compound 80 and pathogen). In FIG. 1, (e) is the results showing the degrees of the defense genes expressions generated after 12 hours since Compound 89 was treated and Phytophthora capcisi was inoculated (1. Control group, 0 hour; 2. Control group, 12 hours; 3. 0.1 mM of BTH, 0 hour; 4. 0.1 mM of BTH, 12 hours; 5. 0.1 ppm of Compound 89, 0 hour; 6. 0.1 ppm of Compound 89, 12 hours; 7. 1.0 ppm of Compound 89, 0 hour; 8. 1.0 ppm of Compound 89, 12 hours).

FIG. 2 is the photographs illustrating the expression degree (X-gluc histochemical staining) of GUS generated in the case where Compound 74 was treated to a tobacco plant.

FIG. 3 is the photographs illustrating the effects on suppressing a tobacco bacterial rot (Pectobacterium carotovora) generated in the case where Compound was treated to a tobacco plant. In FIG. 3, (a) is the photograph illustrating the effects on suppressing the bacterial rot in the case of treating Compound 7 (D7). In FIG. 3, (b) is the photographs illustrating the suppression effects in the case of treating Compound 24 (D24), Compound (D27), Compound 37 (D37), Compound 39 (D39), and Compound 40 (D40). In FIG. 3, (c) is the photographs illustrating the suppression effects in the case of treating Compound 51 (D51), Compound 52 (D52), Compound 57 (D57), Compound 59 (D59), and Compound 60 (D60). In FIG. 3, (d) is the photographs illustrating the suppression effects in the case of treating Compound 67 (D67), Compound 68 (D68), Compound 75 (D75), Compound 12 (D12), and Compound 16 (D16).

FIG. 4 is the photographs illustrating the effects on suppressing anthracnose in the case of treating Compound 61 (D61) and Compound 74 (D74) to the cucumber leaves.

FIG. 5 is the photographs illustrating the effects on suppressing (a) a bacterial rot (Pectobacterium carotovora) and (b) phytophthora (Phytophthora capcisi) after Compound (D89) was sprayed to the pepper leaves in the concentrations of 0.1, 1.0, and 10.0 ppm.

FIG. 6 is the photographs illustrating the phenomenon that suppresses the outbreak of a bacterial rot (Pectobacterium carotovora) on the leaves after Compounds 4 (D4), 6 (6), 90 (D90), 91 (D91), 92 (D92), 93 (D93), 94 (D94), and 95 (D95) were respectively drench-treated to the pepper leaves.

FIG. 7 is the photographs illustrating the effects on suppressing the outbreak of a bacterial rot (Pectobacterium carotovora) generated on the leaves after Compounds 6 (D6), (D91), 93 (D93), 95 (D95), and 107 (D107) were respectively sprayed to the pepper leaves.

FIG. 8 is the photographs illustrating the phenomenon that suppresses the outbreak of phytophthora (Phytophthora capcisi) generated on the leaves after Compounds 4 (D4), 94 (D94), 95 (D95), 107 (D107), and 109 (D109) were respectively drench-treated to the pepper leaves.

FIG. 9 is the photographs illustrating the phenomenon that suppresses the outbreak of phytophthora (Phytophthora capcisi) generated on the leaves after Compounds 4 (D4), 6 (D6), 107 (D107), 108 (D108), and 109 (D109) were respectively sprayed to the pepper leaves.

FIG. 10 is the photographs illustrating the degrees of lesion formation on the leaves and roots that were observed after 7 days since Compound 85 was sprayed on the pepper leaves, and then the suspension of phytophthora (Phytophthora capcisi) swarm spore was drench-infected to the roots ((a) Control group; (b) Treatment of 0.1 mM of BTH; (c) Treatment of 10 ppm of Compound 85).

FIG. 11 is the photographs illustrating the degree of cold weather observed on the plants after Compounds 95 (D95), 103 (D103), and 109 (D109) were drench-treated to the pepper plants raised for 6 weeks; after 1 week, the pepper plants were left as it is at a growth chamber of 2° C. for 1 day; and then, the pepper plants were grown at room temperature for 3 days.

FIG. 12 is the photographs illustrating the degree of cold weather observed on the plants after Compounds 4 (D4), (D6), 90 (D90), 91 (D91), 92 (D92), 93 (D93), 94 (D94), and 95 (D95) were sprayed to the leaves of the pepper plants raised for 6 weeks; after 1 week, the pepper plants were left as it is at a growth chamber of 2° C. for 1 day; and then, the pepper plants were grown at room temperature for 3 days.

### BEST MODE

The present invention relates to an agricultural plant-protecting agent including a dipeptide derivative represented by the above-described chemical formula 1 or an agro-pharmaceutically acceptable salt thereof as an active ingredient.

For the dipeptide derivative represented by the above-described chemical formula 1 which is included, as an active ingredient, in the agricultural plant-protecting agent, in the case of having one or more chiral carbons, the agricultural plant-protecting agent of the present invention may include the compound represented by the above-described chemical formula 1 in a type of a racemic mixture or isomeric compound as an active ingredient.

In the present invention, examples of the agro-pharmaceutically acceptable salt may include a metallic salt, a salt with an organic base, a salt with an inorganic acid, a salt with an organic acid, a salt with a basic or acidic amino acid, and the like. Examples of the suitable metallic salt may include an alkali metallic salt, such as, a sodium salt and a potassium salt; an alkali earth metallic salt, such as, a calcium salt, a magnesium salt, and a barium salt; an aluminum salt; and the like. Examples of the salt with an organic base may include the salts with trimethyl amine, triethyl amine, pyridine, picoline, 2,6-lutidine, ethanol amine, diethanol amine, triethanol amine, cyclohexyl amine, dicyclohexyl amine, N,N-dibenzyl ethylene amine, and the like. Examples of the salt with an inorganic acid may include the salts with hydrochloric acid, hydrobromic acid, nitric acid, sulfuric acid, phosphoric acid, and the like. Examples of the salt with an organic acid may include the salt with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, and the like. Examples of the salt with a basic amino acid may include the salts with alginin, lysine, ornithine, and the like. Examples of the salt with an acidic amino acid may include the salts with aspartic acid, glutamic acid, and the like.

The substituent that is used for defining the dipeptide derivative represented by the above-described chemical formula 1 according to the present invention will be described in more detail as follows. The term “alkyl” in the present invention means the linear or branched alkyl group having 1 to 18 carbon atoms, preferably 1 to 6 carbon atoms, and more preferably 1 to 4 carbon atoms. In detail, it may include methyl, ethyl, n-propyl, isopropyl, n-butyl, 1-methyl propyl, 2-methyl propyl, t-butyl, n-pentyl, t-pentyl, n-hexyl, isohexyl, heptyl, octyl, hexadecanyl, octadecanyl, and the like. The term “alkoxy” in the present invention means O-alkyl, and at this time, the alkyl is the same as described above.

In addition, for the dipeptide derivative represented by the above-described Chemical Formula 1, preferably, the R¹, R², and R⁵ are the same or different from each other, and represent a hydrogen atom, an acetyl group, a hexanoyl group, a hexadecanoyl group, an octadecanoyl group, a benzoyl group, a 4-hexylbenzoyl group, a 2-phenylacetyl group, a 3-phenylpropanonyl group, a methoxycarbonyl group, an ethoxycarbonyl group, a t-butoxycarbonyl group, a hexadecanoxy carbonyl group, an octadecanoxycarbonyl group, a phenoxycarbonyl group, and a 4-hexylbenzyloxycarbonyl group; the R³, R⁴, R⁶, and R⁷ are the same or different from each other, and represent a hydrogen atom, a methyl group, an ethyl group, a n-propyl group, an isopropyl group, a 1-methylpropyl group, a 2-methylpropyl group, a hydroxymethyl group, a 1-hydroxyethyl group, a 2-hydroxyethyl group, an imidazole-4-yl-methyl group, a 2-methylthioethyl group, a benzyl group, a 4-hydroxybenzyl group, a phenethyl group, a mercaptomethyl group, a methylthiomethyl group, a methylthioethyl group, a tritylthiomethyl group, a tritylthioethyl group, a methoxycarbonylmethyl group, an ethoxycarbonylmethyl group, a propanoxycarbonylmethyl group, a tert-butoxycarbonylmethyl group, a pentaneoxycarbonylmethyl group, a hexaneoxycarbonylmethyl group, an allyloxycarbonylmethyl group, a 2-allyloxycarbonylethyl group, a benzyloxycarbonylmethyl group, a benzyloxycarbonylethyl group, a penethyloxycarbonylmethyl group, a 2-penethyloxycarbonylethyl group, a 3-phenylpropyloxycarbonylmethyl group, a 2-(3-phenylpropyloxycarbonyl)ethyl group, a 1-methoxycarbonylethyl group, a 2-methoxycarbonylethyl group, a 2-ethoxycarbonylethyl group, a 2-propaneoxycarbonylethyl group, a 2-butoxycarbonylethyl group, a 2-pentaneoxycarbonylethyl group, a 2-hexaneoxycarbonylethyl group, a 2-aminoethyl group, a carbamoylmethyl group, an acetylaminomethyl group, an acetylaminoethyl group, a carboxymethyl group, a carboxyethyl group, an imidazole-4-ylmethyl group, an imidazole-4-ylethyl group, a 3-guanidinopropyl, a N, N-bis(benzyloxycarbonyl)guanidinopropyl, an indole-3-ylmethyl group, or an indole-3-ylethyl group; or any one of the R³ and R⁴ may bind to R² to form a nitrogen-containing 5-membered ring or any one of the R⁶ and R⁷ may bind to R⁵ to form a nitrogen-containing 5-membered ring; and the R⁸ represents the compound representing a hydrogen atom, a methoxy group, an ethyoxy group, a propaneoxy group, a t-butoxy group, a hexaneoxy group, a hexadecaneoxy group, an octadecaneoxy group, a benzyloxy group, a penethyloxy group, a 3-phenylpropaneoxy group, a methylamino group, an ethylamino group, a propylamino group, a butylamino group, a hexylamino group, a phenylamino group, a benzylamino group, a hexadecylamino group, and an octadecylamino group.

In addition, the dipeptide derivative represented by the above-described chemical formula 1 is the same in more detail as follows:
methyl 2-(2-((t-butoxycarbonyl)amino)-3-hydroxypropanamido)-4-methylpentanoate;
methyl 2-(2-amino-3-hydroxypropanamido)-4-methylpentanoate;
methyl 2-(2-((t-butoxycarbonyl)amino)-4-methylpentanamido)acetate;
methyl 2-(2-amino-4-methylpentanamido)acetate;
methyl 2-(2-((t-butoxycarbonyl)amino)-3-methylbutanamido)acetate;
methyl 2-(2-amino-3-methylbutanamido)acetate;
methyl 2-(2-((t-butoxycarbonyl)amino)-3-hydroxybutanamido)-4-methylpentanoate;
methyl 2-(2-amino-3-hydroxybutanamido)-4-methylpentanoate;
ethyl 2-(2-((t-butoxycarbonyl)amino)-4-methylpentanamido)propanoate;
ethyl 2-(2-amino-4-methylpentanamido)propanoate;
2-(2-amino-4-methylpentanamido)propanoic acid;
methyl 2-(2-((t-butoxycarbonyl)amino)-3-methylbutanamido)propanoate;
methyl 2-(2-((t-butoxycarbonyl)amino)-propanamido)propanoate;
Methyl 2-(2-((t-butoxycarbonyl)amino)-ethanamido)propanoate;
methyl 2-(2-((t-butoxycarbonyl)amino)-3-((4-hydroxyphenyl)propanamido)propanoate;
methyl 2-(2-((t-butoxycarbonyl)amino)-acetic propanamido)-3-(indole-3yl)propanoate;
methyl 2-(2-((t-butoxycarbonyl)amino)-3-methylbutanamido)-3-(indole-3yl)propanoate;
methyl 1-(2-((t-butoxycarbonyl)amino)-3-phenylpropane oil)pyrolidine-2-carboxylate;
methyl 1-(2-((t-butoxycarbonyl)amino)propane oil)pyrolidine-2-carboxylate;
methyl 1-(2-((t-butoxycarbonyl)amino)-3-methylbutane oil)pyrolidine-2-carboxylate;
methyl 1-(2-((t-butoxycarbonyl)amino)-4-methylpentane oil)pyrolidine-2-carboxylate;
methyl 1-(5-amino-2-((t-butoxycarbonyl)amino)-5-oxopentane oil)pyrolidine-2-carboxylate;
methyl 1-(2-((t-butoxycarbonyl)amino)-3-hydroxybutane oil)pyrolidine-2-carboxylate;
methyl 1-(4-t-butoxy-2-((t-butoxycarbonyl)amino)-4-oxobutane oil)pyrolidine-2-carboxylate;
methyl 1-(4-amino-2-((t-butoxycarbonyl)amino)-4-oxobutane oil)pyrolidine-2-carboxylate;
t-butyl 2-((2-methoxycarbonyl)pyrolidine-1-carbonyl)pyrolidine-1-carboxylate;
methyl 1-((2,6-bis(t-butoxycarbonyl)amino)hexane oil)pyrolidine-2-carboxylate;
methyl 1-(2-((t-butoxycarbonyl)amino)-3-(imidazole-4-yl)propane oil)pyrolidine-2-carboxylate;
methyl 1-(5-t-butoxy-2-((t-butoxycarbonyl)amino)-5-oxopentane oil)pyrolidine-2-carboxylate;
methyl 1-(2-((t-butoxycarbonyl)amino)-3-(4-hydroxyphenyl)propanoyl)pyrolidine-2-carboxylate;
methyl 1-(2-((t-butoxycarbonyl)amino)-3-methylpentane oil)pyrolidine-2-carboxylate;
methyl 1-(2-((t-butoxycarbonyl)amino)acetyl)pyrolidine-2-carboxylate;
methyl 1-(2-((t-butoxycarbonyl)amino)-3-hydroxypropane oil)pyrolidine-2-carboxylate;
methyl 1-(2-((t-butoxycarbonyl)amino)-4-methylthiobutane oil)pyrolidine-2-carboxylate;
methyl 1-(2-((t-butoxycarbonyl)amino)-3-(indole-3-yl)propane oil)pyrolidine-2-carboxylate;
methyl 1-(2-((t-butoxycarbonyl)amino)-3-trithylthiopropane oil)pyrolidine-2-carboxylate;
methyl 1-(2-((t-butoxycarbonyl)amino)-3-mercaptopropane oil)pyrolidine-2-carboxylate;
methyl 2-(2-((t-butoxycarbonyl)amino)-4-methylpentanamido))-3-hydroxybutanoate;
ethyl 2-(2-((t-butoxycarbonyl)amino)-3-methylpentanamido))-propanoate;
methyl 2-(2-((t-butoxycarbonyl)amino)-3-hydroxypropanamido))-acetate;
ethyl 2-(2-((t-butoxycarbonyl)amino)-ethanamido))-propanoate;
methyl 2-(2-((t-butoxycarbonyl)amino)-4-methylpentanamido))-3-phenylpropanoate;
methyl 2-(2-((t-butoxycarbonyl)amino)-ethanamido)-3-methylbutanoate;
methyl 2-(2-amino-ethanamido)-3-methylbutanoate;
3-(1-(methoxycarbonyl)-2-phenylethylcarbamoyl)-3-aminopropanoic acid;
methyl 3-(1-methoxycarbonyl-2-phenylethylcarbamoyl)-3-(t-butoxycarbonylamino)propanoate;
methyl 3-(1-(methoxycarbonyl)-2-phenylethylcarbamoyl)-3-aminopropanoate;
allyl 3-(1-(methoxycarbonyl)-2-phenylethylcarbamoyl)-3-(N-butoxycarbonylamino)propanoate;
allyl 3-(1-(methoxycarbonyl)-2-phenylethylcarbamoyl)-3-aminopropanoate;
benzyl 3-(1-(methoxycarbonyl)-2-phenylethylcarbamoyl)-3-(N-butoxycarbonylamino)propanoate;
benzyl 3-(1-(methoxycarbonyl)-2-phenylethylcarbamoyl)-3-aminopropanoate;
methyl 3-(1-(methoxycarbonyl)-2-phenylethylcarbamoyl)-3-(N-acetylamino)propanoate;
methyl 3-(1-(methoxycarbonyl)-2-phenylethylcarbamoyl)-3-(N-benzoylamino)propanoate;
allyl 3-(1-(methoxycarbonyl)-2-phenylethylcarbamoyl)-3-(N-acetylamino)propanoate;
allyl 3-(1-(methoxycarbonyl)-2-phenylethylcarbamoyl)-3-(N-benzoylamino)propanoate;
benzyl 3-(1-(methoxycarbonyl)-2-phenylethylcarbamoyl)-3-(N-acetylamino)propanoate;
benzyl 3-(1-(methoxycarbonyl)-2-phenylethylcarbamoyl)-3-(N-benzoylamino)propanoate;
3-(1-(methoxycarbonyl)-2-phenylethylcarbamoyl)-3-(N-acetylamino)propanoic acid;
methyl 2-(2-acetylamino)-3-methylbutanamido)acetate;
methyl 2-(2-acetylamino)-4-methylpentanamido)acetate;
methyl 2-((2-pyrolidinecarbamoyl)amino)acetate;
methyl 1-(2-amino-3-(indole-3-yl)propanoyl)pyrolidine-2-carboxylate;
methyl 2-(2-amino-3-methylbutanamido)propanoate;
methyl 2-(2-amino-3-methylpentanamido)propanoate; and
the agro-pharmaceutically acceptable salts thereof.

The dipeptide derivative represented by the above-described chemical formula 1 is an intermediate for synthesizing a 2,5-diketopiperazine compound, and the method for preparing the same is already known in many literatures [Akiyama et al, J. Chem. Soc., Perkin Trans. 1 1989, 235; Gordon et al, Bioorg. Med. Chem. Lett., 1995, 5, 47; Carlsson, A. C. Tetrahedron Lett. 2006, 47, 5199; Lopez-Cobenas, A. Synlett. 2005, 1158; Boehm et al, J. Org. Chem. 1986, 51, 2307; Thajudeen et al, Tetrahedron Lett., 2010, 51, 1303]. Therefore, the person who skilled in the organic synthesis field can easily synthesize the dipeptide derivative represented by the above-described chemical formula 1 with reference to the known literatures.

The dipeptide derivative represented by the above-described chemical formula 1 or agro-pharmaceutically acceptable salt thereof can exhibit a resisting effect on various plant diseases caused by bacteria, virus, and molds by inducing the production of plant disease-preventing proteins through expressing the resistance genes in a plant. Therefore, it is useful as an active ingredient of the agricultural plant-protecting agent. In detail, the plant diseases caused by bacteria, virus, and molds may include a bacterial rot, damping-off, phytophthora blight, a wilt disease, a leaf spot disease or a mosaic disease of a plant. The plants, to which the agricultural plant-protecting agent including the dipeptide derivative represented by the above-described chemical formula 1 as an active ingredient is applied, may be dicotyledoneae, for example, solanaceae such as potatoes, peppers, sweet peppers, and tomatoes, cucurbitaceae, such as cucumbers, cigarettes, watermelons, and oriental melons, a crucifer (cruciferae) such as Chinese cabbage, lettuces, radishes, cabbages, rapes, peanuts, and a salary, a medicinal plant, such as ginseng and dong quai, a plant, such as perilla seeds, strawberry, spring onion, garlic, ginger, and onion, graminae, such as, rice, barley, corn, and sorghum, and fruit trees, such as apple tree, pear tree, peach tree, and persimmon tree. It is preferable to apply to dicotyledoneae, such as, cigarettes, Chinese cabbage, peppers, strawberry, cucumbers, potatoes, and tomatoes.

The agricultural plant-protecting agent of the present invention may include, as an active ingredient, singly, the dipeptide derivative represented by the above-described chemical formula 1 or an agro-pharmaceutically acceptable salt. In addition, the agricultural plant-protecting agent of the present invention includes 0.001 to wt %, and preferably 0.005 to 30 wt % of the active ingredient, and may further include excipient, residually. The excipient may be a general microorganism formulation, an antibacterial effect-promoting agent, diluents, or carrier. In addition to the agricultural active ingredient of the present invention, other germicides, insecticides, herbicides, plant growth-controlling agents or fertilizers, which are conventionally commercialized and used, or developed for increasing a remedial effect or expanding the range of application, are further included in the range of certain content, and thus, may be formulated in a type of mixed formulation.

The excipient and dilutent included in the agricultural plant-protecting agent of the present invention are generally used in the agricultural field, and may be included by further adding solid carriers, for example, oxides, such as, diatomite and slaked lime, phosphate, such as, apatite, a sulfate, such as, gypsum, and mineral powders, such as, clay, kaolin, bentonite, acid clay, quartz, and silica, and filler, anti-aggregating agents, a surfactant, an emulsifying agent, and preservatives thereto. In addition, the agricultural drugs of the present invention may be formulated in order to release the active ingredient in the types of prompt release, sustained release, and delayed release by using the method that are known in the art. For the formulation, additives, such as, the surfactant, diluent, dispersant, and adjuvants that are generally used are mixed with the active ingredient, and then, the mixture thus obtained may be formulated in various types, such as, water dispersible powders, suspension, emulsion, emulsifiable concentrate, microemulsion, liquid formulation, dispersible formulation, particulate water dispersible powders, granule, powdered medicine, liquid water dispersible powder, particulate water dispersible powder, water floating granule, and tablets, and then used.

The agricultural plant-protecting agent of the present invention may be applied to a plant by using the general method. For applying it to a plant, it may be directly scattered or applied to leaves, stems, branches, roots, and seeds of plant body; may be mixed with the general cultivation soils, such as, a paddy field or dry field or nursery bed soil or media; or, in the case of the plant body cultured in water, may be treated to the surface of water in order to prevent the damage of disease. As a specific applying method, there may be an application treatment, a dipping treatment, a fumigation treatment, or a scattering treatment. For example, the agricultural plant-protecting agent may be scattered to soils, or the leaves, stems, seeds, flowers, or fruits of a plant. In order to apply the agricultural plant-protecting agent of the present invention to a plant, the agent may be diluted with water or proper medium, and then used.

In order to confirm the plant disease inducible resistance (ISR) effect, the plant growth-promoting effect, and cold weather-overcoming effect, multidirectional experiments were performed.

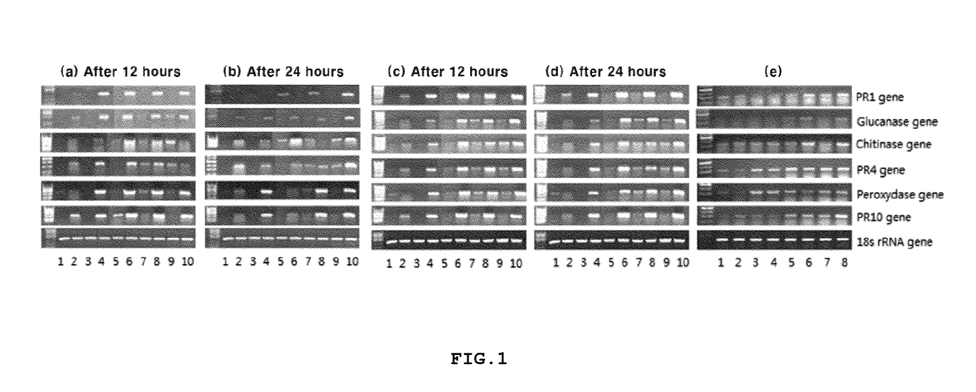

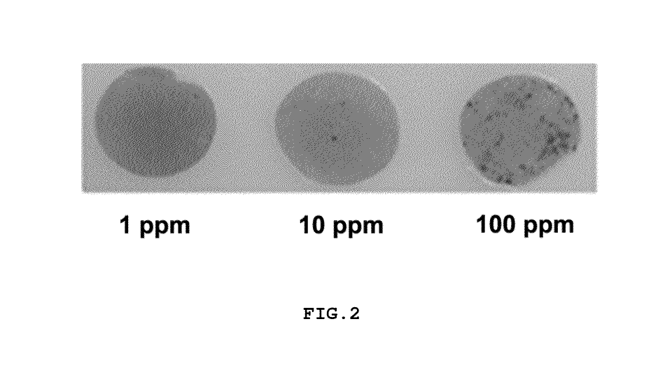

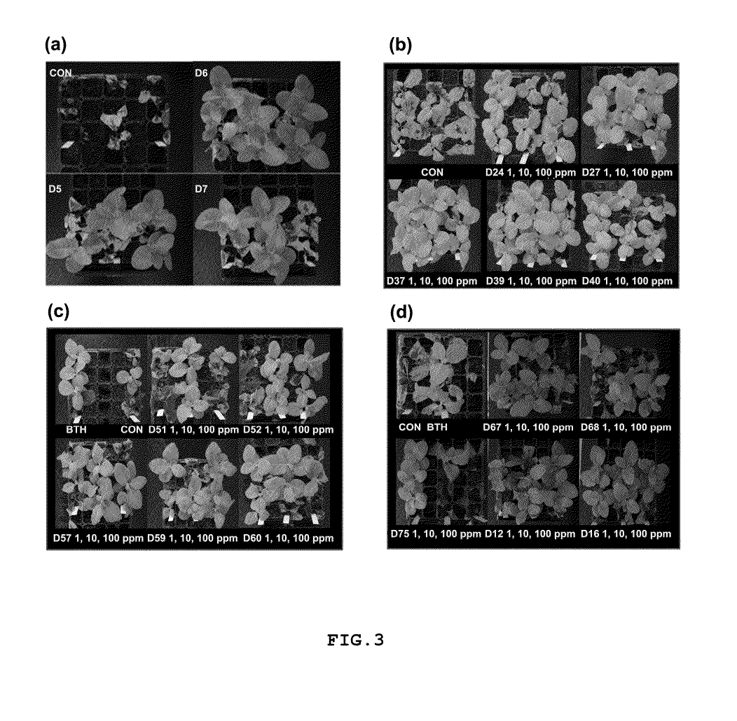

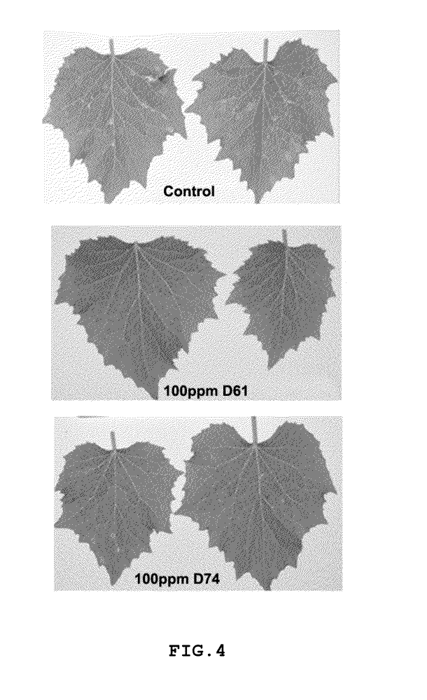

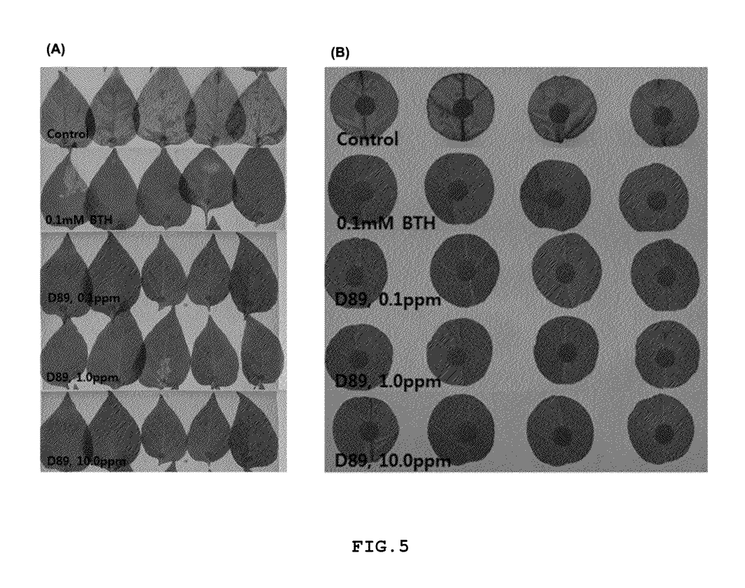

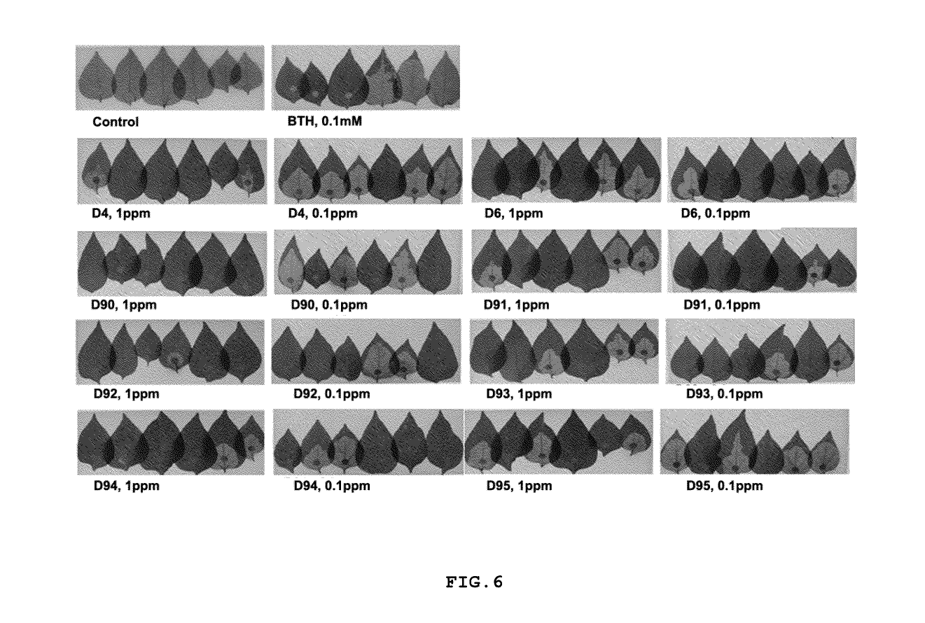

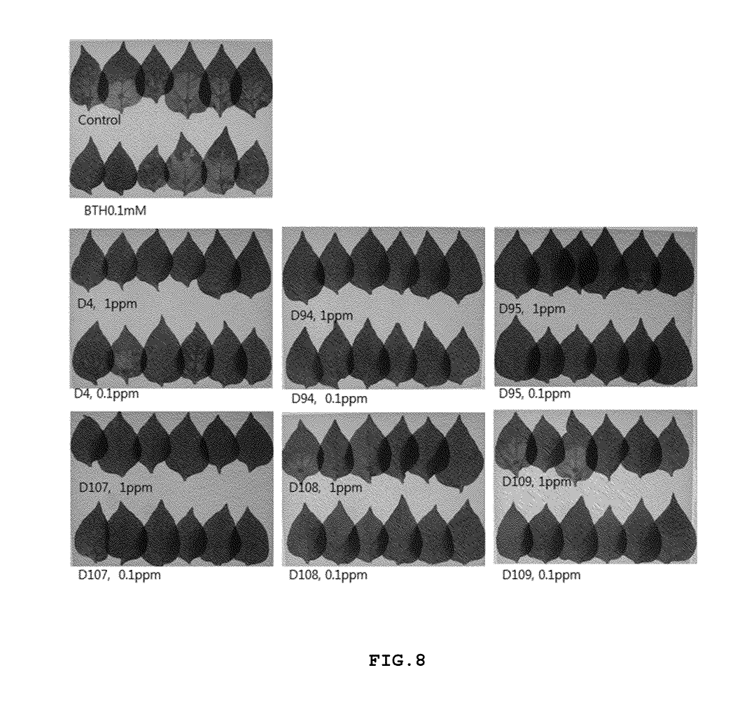

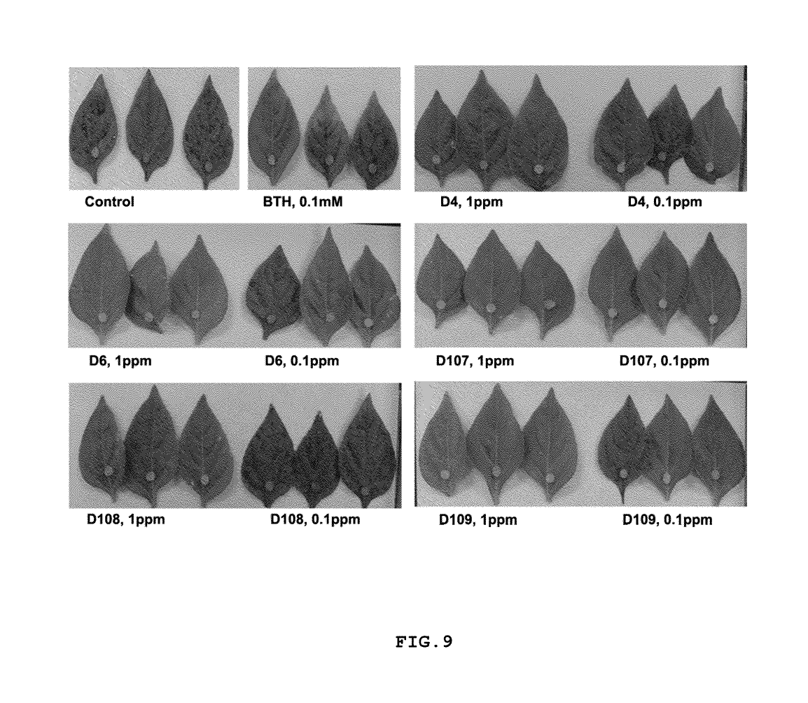

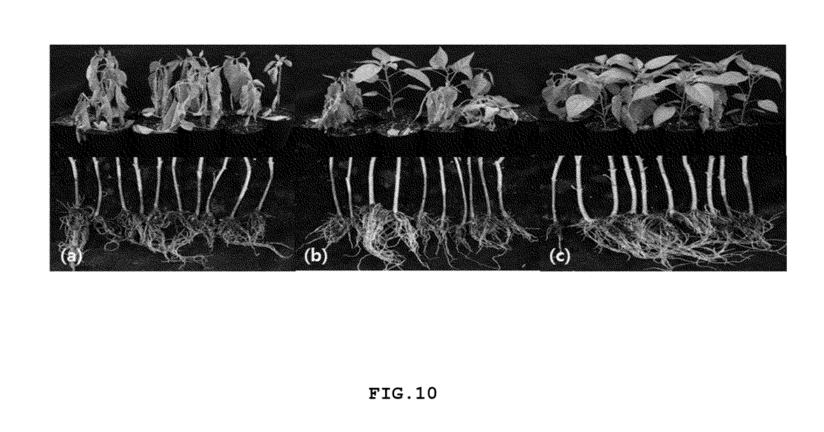

First, a dipeptide derivative of the present invention was treated to a pepper plant, and then the degrees of the expressions of the main defense genes, for example, antibiotic genes, such as, PR-1, beta-1,3-glucanase, chitinase, PR4, peroxidase, and PR10 primer were measured. It was confirmed that for the test groups treated with the dipeptide derivative represented by the above-described chemical formula 1, the expressions of the genes suppressing the plant disease outbreak were significantly increased as compared with the control group. In addition, it was also confirmed that the defense genes were not expressed by treatment only with the agents before being inoculated with the plant pathogen, but the resistance mechanism started to operate in the plant infected with the plant pathogen, such as, the pathogen of a bacterial rot (Pectobacterium carotovorum SCC1) and the pathogen of phytophthora (Phytophthora capcisi) (see FIG. 1). Therefore, it can be found that the agricultural plant-protecting agent of the present invention has excellent effect on suppressing the infection and propagation of the pathogen, and thus, preventing or treating the outbreak of plant disease. As a result of treating the dipeptide derivative of the present invention to the tobacco leaves bound with GUS gene induced by a PR-1α disease resistance promoter, it can be confirmed that the PR-1α GUS activity is significantly increased as compared with the control group (see FIG. 2). In addition, in order to confirm the effect on suppressing the outbreak of the plant disease by treating the agricultural plant-protecting agent of the present invention to a plant, the effects of the solution of the dipeptide derivative represented by the above-described chemical formula 1 on suppressing a bacterial rot or anthracnose were tested to the plants, such as, cigarette, Chinese cabbage, cucumbers, peppers, and cabbage as an object. It could be confirmed that when the dipeptide derivative represented by the above-described chemical formula 1 was treated to a plant, the effect thereof on suppressing the outbreak of the plant disease was significantly improved as compared with the non-treated group or control drug (BTH)-treated group (see FIG. 3). From the result of observing the degree of the suppression of the plant disease outbreak on the leaves of the plant prepared by treating the dipeptide derivative to the seeds, and then growing the seeds, it could be confirmed that the lesion formation was largely suppressed as compared with the non-treated group (see FIG. 4). For this reason, it can be confirmed that when the dipeptide derivative of the present invention is treated to the leaves or stems, the effect on suppressing the lesion is excellent, and also, even when it is treated to the seeds, the lesion formation can be suppressed. It could be confirmed that when Compounds 89, 91, 93, and 107, that are the dipeptide derivative according to the present invention, were directly sprayed to the leaves of the plant after the plant was infected with the plant pathogen, the effect on suppressing the outbreak of pepper phytophthora blight or bacterial rot was excellent as compared with the non-treated control group (see Table 7, and FIGS. 5, 7, and 9). In addition, it could be confirmed that when Compounds 4, 94, 95, and 107, which are the dipeptide compound according to the present invention, were drench-treated to the roots of the plant, the outbreak of the phytophthora blight on the leaves was significantly decreased (see FIGS. 6 and 8). In addition, when the dipeptide compounds according to the present invention were sprayed to the leaves of the pepper plant, the roots of the plant were infected with the pepper phytophthora (Phytophthora capsici), and then the lesion formations were observed on the roots and leaves, the lesion formations were significantly decreased on the leaves and roots as compared with the control group (see FIG. 10).

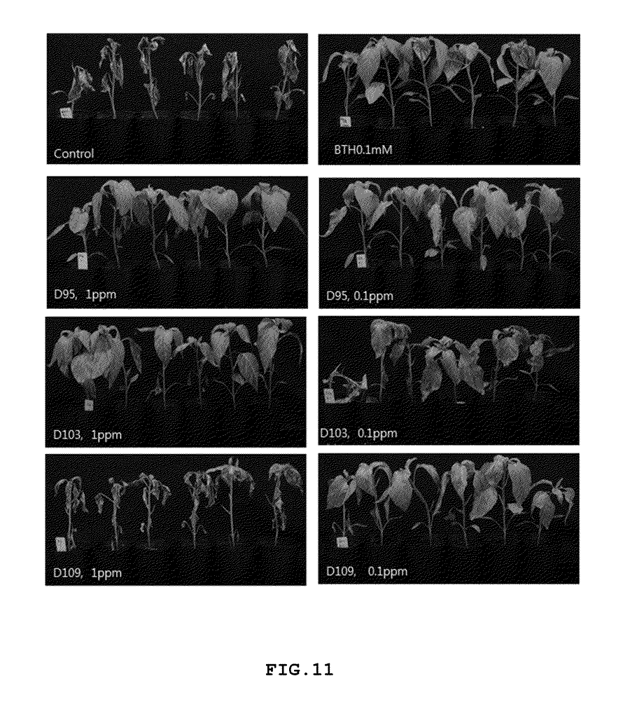

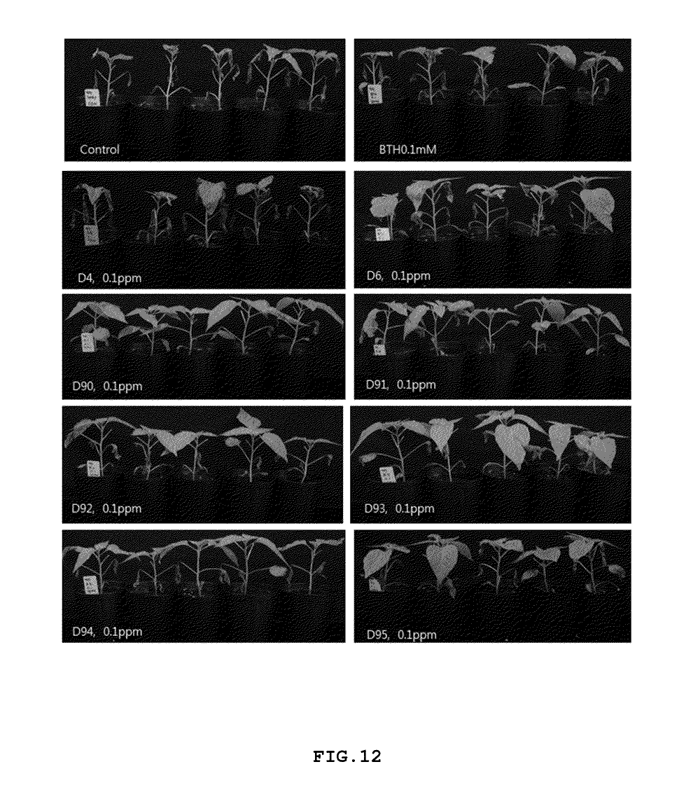

In addition, it could be confirmed that when the agricultural plant-protecting agent of the present invention was treated to the plant, the plant growth was promoted up to the maximum 24.4% as compared with the non-treated group (see FIG. 6). The dipeptide compound according to the present invention exhibits the excellent effect on preventing the cold-weather damage of the plant, and when the leaves that were drench-treated or sprayed with the dipeptide compound were exposed under a low temperature for a certain period of time to be subjected to the cold-weather damage, the leaves exhibited the excellent ability of overcoming the cold-weather damage and then being restored in its original condition (see FIGS. 11 and 12).

As described above, the agricultural plant-protecting agent of the present invention can induce the disease resistance of the plant; also, can exhibit the effect on promoting the growth; and can exhibit the effect to endure the physical stress such as the cold-weather damage. Therefore, the agent of the present invention has very high value for being applied as the next generation agricultural plant-protecting agent having the action mechanism that is completely different from the conventional plant-protecting agents. The plant disease-preventing effect, plant growth-promoting effect, and plant immunity-activating effect (plant disease-resisting effect and cold-weather damage-preventing effect) by the agricultural plant-protecting agent of the present invention will be described in more detail in the following Examples.

As described above, the present invention will be described in more detail with reference to the following Synthesis Examples and Examples, but the present invention is not limited thereto.

### Synthesis Example

### Representative Synthesis Example

### Synthesis of Dipeptide Derivative

(1) Dipeptide Synthesis Method A (Compounds 1, 3, 5, 7, 9, 12 to 24, 26, 27, 29, 32 to 36, 38 to 47, 49, 51 to 60, 62 to 73, 75, 76, 79 to 87, and 106)

Various L- or D-type N-Boc-amino acids (3.0 mmol) were dissolved in 10 mL of dimethylformamide (DMF). Diisopropylmethylamide (DIEA; 0.78 g, 6.0 mmol), 0-(benzotriazole-1-yl)-N,N,N,N-tetramethyluronium hexafluorophosphate (HBTU′ 1.37 g, 3.6 mmol) was added thereto, and then stirred for about 30 minutes. L- or D-type amino acid alkyl (methyl or ethyl) ester (hydrochloride) (3.6 mmol) was added to the reaction mixture thus obtained, and then stirred at room temperature for 12 hours. The reaction mixture thus obtained was diluted with ethyl acetate, and then washed with sodium hydrogen carbonate or salt water. Water was removed from the organic layer with anhydride sodium sulfate, and then the organic layer without water was concentrated under reduced pressure. The concentrated solution thus obtained was purified through a silica column chromatograph (EtOAc/Hexane) to obtain linear N-Boc-L-amino acid-L-amino acid alkyl ester. The yield rate of the condensation reaction was about 50% to 95%.

(2) Dipeptide Synthesis Method B (Compounds 25, 28, 30, 31, 37, 48, 50, 61, 74, 77, 90, 92, and 94)

By using 10.0 mmol of the cysteine, in which a SH group of the cysteine is protected with a trityl group, the aspartic acid or glutamic acid derivative, in which its terminal COOH group is converted with alkyl ester, and lysine or arginine, in which its terminal amino group is protected with a Boc or benzyloxycarbonyl group, the linear N-Boc-L-amino acid-L-amino acid alkyl esters were synthesized by using the same method as Synthesis method A described above. The yield rate of the condensation reaction was about 65% to 80%.

(3) Dipeptide Synthesis Method C (Compounds 2, 4, 6, 8, 10, 11, 78, 88, 89, 91, 93, 95, and 107 to 109)

The N-Boc-amino acid-amino acid alkyl esters (5 mmol) obtained from Synthesis method A and B were dissolved in 50% trifluoroacetic acid-methylene chloride solution (20 mL), and then stirred for 2 hours. The solvent was evaporated under reduced pressure to obtain the amino acid-amino acid alkyl ester trifluoroacetic acid salt without a protecting group, such as, Boc or trityl. As another method, N-Boc-amino acid-amino acid methyl ester (5 mmol) was dissolved in a formic acid (10 mL), stirred for 6 hours, and then vacuum-concentrated to obtain the amino acid-amino acid alkyl ester formic acid without a protecting group. Compound 89 was purchased from Sigma-Aldrich and then used. Compounds 91, 93, and 95 could be obtained from Synthesis method described above, and also could be obtained from Compound 89 by reacting with methyl iodide, allyl bromide, and benzyl chloride, respectively, in MeCN/MeOH.

(4) Dipeptide Synthesis Method D (Compound 11)

The N-Boc-amino acid-amino acid alkyl esters obtained from Synthesis method A and B were again dissolved in water-soluble dioxane, the equivalent LiOH was added thereto, and then the mixture thus obtained was stirred overnight. Since then, the mixture thus obtained was neutralized with acetic acid and was extracted three times with the mixed solvent of acetone/ethanol to obtain the amino acid-amino acid without an alkyl group.

(5) Dipeptide Synthesis Method E (Compounds 96 to 105)

The amino acid-amino acid alkyl ester salt (0.57 mmol) obtained from Synthesis method C was dissolved in methylene chloride (5 mL), acyl chloride (1.2 eq) and triethyl amine (2.4 eq) were added thereto, and then the reaction mixture thus obtained was stirred at room temperature for 2 hours. The reaction mixture thus obtained was added to EtOAc (50 mL), washed with 1 M HCl solution, sodium carbonate, and saturated salt water, and then concentrated. The concentrated solution thus obtained was purified through a silica column chromatograph (EtOAc/Hexane) to obtain N-acyl amino acid-amino acid alkyl ester in the yield rate of 90% or more.

The compounds prepared by using the methods of the representative Synthesis Examples are listed in the following Table 1.

TABLE 1

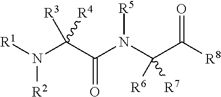

| Com- | | | | | | | | |
|---|---|---|---|---|---|---|---|---|
| pound | | | | | | | | |
| No. | R¹ | R² | R³ | R⁴ | R⁵ | R⁶ | R⁷ | R⁸ |
| 1 | (CH₃)₃COOC— | H— | HOCH₂— | (S) | H— | (CH₃)₂CHCH₂— | (S) | MeO— |
| | | | | H— | | | H— | |
| 2 | H— | H— | HOCH₂— | (S) | H— | (CH₃)₂CHCH₂— | (S) | MeO— |
| | | | | H— | | | H— | |
| 3 | (CH₃)₃COOC— | H— | (CH₃)₂CHCH₂— | (S) | H— | H— | H— | MeO— |
| | | | | H— | | | | |
| 4 | H— | H— | (CH₃)₂CHCH₂— | (S) | H— | H— | H— | MeO— |
| | | | | H— | | | | |
| 5 | (CH₃)₃COOC— | H— | (CH₃)₂CH— | (S) | H— | H— | H— | MeO— |
| | | | | H— | | | | |
| 6 | H— | H— | (CH₃)₂CH— | (S) | H— | H— | H— | MeO— |
| | | | | H— | | | | |
| 7 | (CH₃)₃COOC— | H— | CH₃CH(OH)— | (S) | H— | (CH₃)₂CHCH₂— | (S) | MeO— |
| | | | | H— | | | H— | |
| 8 | H— | H— | CH₃CH(OH)— | (S) | H— | (CH₃)₂CHCH₂— | (S) | MeO— |
| | | | | H— | | | H— | |
| 9 | (CH₃)₃COOC— | H— | (CH₃)₂CHCH₂— | (S) | H— | CH₃— | (S) | EtO— |
| | | | | H— | | | H— | |
| 10 | H— | H— | (CH₃)₂CHCH₂— | (S) | H— | CH₃— | (S) | EtO— |
| | | | | H— | | | H— | |
| 11 | H— | H— | (CH₃)₂CHCH₂— | (S) | H— | CH₃— | (S) | HO— |
| | | | | H— | | | H— | |
| 12 | (CH₃)₃COOC— | H— | (CH₃)₂CH— | (S) | H— | CH₃— | (S) | MeO— |
| | | | | H— | | | H— | |
| 13 | (CH₃)₃COOC— | H— | CH₃— | (S) | H— | CH₃— | (S) | MeO— |
| | | | | H— | | | H— | |
| 14 | (CH₃)₃COOC— | H— | H— | H— | H— | CH₃— | (S) | MeO— |
| | | | | | | | H— | |
| 15 | (CH₃)₃COOC— | H— | (4-HO—C₆H₄)— | (S) | H— | CH₃— | (S) | MeO— |
| | | | CH₂— | H— | | | H— | |
| 16 | (CH₃)₃COOC— | H— | H— | (S) | H— | Indole-3-yl- | (S) | MeO— |
| | | | | H— | | CH₂— | H— | |
| 17 | (CH₃)₃COOC— | H— | (CH₃)₂CH— | (S) | H— | Indole-3-yl- | (S) | MeO— |
| | | | | H— | | CH₂— | H— | |
| 18 | (CH₃)₃COOC— | H— | H— | H— | H— | H— | H— | MeO— |
| Com- | | | | | | | | |
| pound | | | | | | | | |
| No. | R¹ | R² | R³ | R⁴ | R⁵-R⁶ | | R⁷ | R⁸ |
| 19 | (CH₃)₃COOC— | H— | C₆H₅—CH₂— | (S) | —CH₂CH₂CH₂— | | (S) | MeO— |
| | | | | H— | | | H— | |
| 20 | (CH₃)₃COOC— | H— | CH₃— | (S) | —CH₂CH₂CH₂— | | (S) | MeO— |
| | | | | H— | | | H— | |
| 21 | (CH₃)₃COOC— | H— | (CH₃)₂CH— | (S) | —CH₂CH₂CH₂— | | (S) | MeO— |
| | | | | H— | | | H— | |
| 22 | (CH₃)₃COOC— | H— | (CH₃)₂CHCH₂— | (S) | —CH₂CH₂CH₂— | | (S) | MeO— |
| | | | | H— | | | H— | |
| 23 | (CH₃)₃COOC— | H— | NH₂COCH₂CH₂— | (S) | —CH₂CH₂CH₂— | | (S) | MeO— |
| | | | | H— | | | H— | |
| 24 | (CH₃)₃COOC— | H— | CH₃(OH)CH | (S) | —CH₂CH₂CH₂— | | (S) | MeO— |
| | | | | H— | | | H— | |
| 25 | (CH₃)₃COOC— | H— | t-BuOOCCH₂— | (S) | —CH₂CH₂CH₂— | | (S) | MeO— |
| | | | | H— | | | H— | |
| 26 | (CH₃)₃COOC— | H— | NH₂COCH₂— | (S) | —CH₂CH₂CH₂— | | (S) | MeO— |
| | | | | H— | | | H— | |
| 27 | (CH₃)₃COOC— | —CH₂CH₂CH₂— | | (S) | —CH₂CH₂CH₂— | | (S) | MeO— |
| | | | | H— | | | H— | |
| 28 | (CH₃)₃COOC— | H— | (CH₃)₃CCOOCNH | (S) | —CH₂CH₂CH₂— | | (S) | MeO— |
| | | | (CH₂)₄— | H— | | | H— | |
| 29 | (CH₃)₃COOC— | H— | Imidazole-4- | (S) | —CH₂CH₂CH₂— | | (S) | MeO— |
| | | | yl-CH₂— | H— | | | H— | |
| 30 | (CH₃)₃COOC— | H— | t- | (S) | —CH₂CH₂CH₂— | | (S) | MeO— |
| | | | BuOOCCH₂CH₂— | H— | | | H— | |
| 31 | (CH₃)₃COOC— | H | N,N— | (S) | —CH₂CH₂CH₂— | | (S) | MeO— |
| | | | (C₆H₅CH₂OOC)₂- | H— | | | H— | |
| | | | guanidinyl- | | | | | |
| | | | (CH₂)₃— | | | | | |
| 32 | (CH₃)₃COOC— | H— | (4-HO-C₆H₄)— | (S) | —CH₂CH₂CH₂— | | (S) | MeO— |
| | | | CH₂— | H— | | | H— | |
| 33 | (CH₃)₃COOC— | —CH₂CH₂CH₂— | | (S) | —CH₂CH₂CH₂— | | (R) | MeO— |
| | | | | H— | | | H— | |
| 34 | (CH₃)₃COOC— | H— | CH₃CH₂(CH₃)CH— | (S) | —CH₂CH₂CH₂— | | (R) | MeO— |
| | | | | H— | | | H— | |
| 35 | (CH₃)₃COOC— | H— | (4-HO—C₆H₄)— | (S) | —CH₂CH₂CH₂— | | (R) | MeO— |
| | | | CH₂— | H— | | | H— | |
| 36 | (CH₃)₃COOC— | H— | (CH₃)₂CHCH₂— | (S) | —CH₂CH₂CH₂— | | (R) | MeO— |
| | | | | H— | | | H— | |
| 37 | (CH₃)₃COOC— | H— | t-BuOOCCH₂— | (S) | —CH₂CH₂CH₂— | | (R) | MeO— |
| | | | | H— | | | H— | |
| 38 | (CH₃)₃COOC— | H— | H— | H— | —CH₂CH₂CH₂— | | (R) | MeO— |
| | | | | | | | H— | |
| 39 | (CH₃)₃COOC— | H— | HOCH₂— | (S) | —CH₂CH₂CH₂— | | (R) | MeO— |
| | | | | H— | | | H— | |
| 40 | (CH₃)₃COOC— | H— | CH₃SCH₂CH₂— | (S) | —CH₂CH₂CH₂— | | (R) | MeO— |
| | | | | H— | | | H— | |
| 41 | (CH₃)₃COOC— | H— | NH₂COCH₂— | (S) | —CH₂CH₂CH₂— | | (R) | MeO— |
| | | | | H— | | | H— | |
| 42 | (CH₃)₃COOC— | H— | C₆H₅—CH₂— | (S) | —CH₂CH₂CH₂— | | (R) | MeO— |
| | | | | H— | | | H— | |
| 43 | (CH₃)₃COOC— | H— | Indole-3-yl- | (S) | —CH₂CH₂CH₂— | | (R) | MeO— |
| | | | CH₂— | H— | | | H— | |
| 44 | (CH₃)₃COOC— | H— | CH₃— | (S) | —CH₂CH₂CH₂— | | (R) | MeO— |
| | | | | H— | | | H— | |
| 45 | (CH₃)₃COOC— | H— | NH₂COCH₂CH₂— | (S) | —CH₂CH₂CH₂— | | (R) | MeO— |
| | | | | H— | | | H— | |
| 46 | (CH₃)₃COOC— | H— | Imidazole-4- | (S) | —CH₂CH₂CH₂— | | (R) | MeO— |
| | | | yl-CH₂— | H— | | | H— | |
| 47 | (CH₃)₃COOC— | H— | CH₃CH(OH)— | (S) | —CH₂CH₂CH₂— | | (R) | MeO— |
| | | | | H— | | | H— | |
| 48 | (CH₃)₃COOC— | H— | (CH₃)₃CCOOCNH | (S) | —CH₂CH₂CH₂— | | (R) | MeO— |
| | | | (CH₂)₄— | H— | | | H— | |
| 49 | (CH₃)₃COOC— | H— | (CH₃)₂CH— | (S) | —CH₂CH₂CH₂— | | (R) | MeO— |
| | | | | H— | | | H— | |
| 50 | (CH₃)₃COOC— | H— | t- | (S) | —CH₂CH₂CH₂— | | (R) | MeO— |
| | | | BuOOCCH₂CH₂— | H— | | | H— | |
| 51 | (CH₃)₃COOC— | H— | CH₃— | (R) | —CH₂CH₂CH₂— | | (S) | MeO— |
| | | | | H— | | | H— | |
| 52 | (CH₃)₃COOC— | H— | Indole-3-yl-CH₂— | (R) | —CH₂CH₂CH₂— | | (S) | MeO— |
| | | | | H— | | | H— | |
| 53 | (CH₃)₃COOC— | H— | (4-HO—C₆H₄)— | (R) | —CH₂CH₂CH₂— | | (S) | MeO— |
| | | | CH₂— | H— | | | H— | |
| 54 | (CH₃)₃COOC— | H— | NH₂COCH₂CH₂— | (R) | —CH₂CH₂CH₂— | | (S) | MeO— |
| | | | | H— | | | H— | |
| 55 | (CH₃)₃COOC— | —CH₂CH₂CH₂— | | (R) | —CH₂CH₂CH₂— | | (S) | MeO— |
| | | | | H— | | | H— | |
| 56 | (CH₃)₃COOC— | H— | (CH₃)₂CH— | (R) | —CH₂CH₂CH₂— | | (S) | MeO— |
| | | | | H— | | | H— | |
| 57 | (CH₃)₃COOC— | H— | (CH₃)₂CHCH₂— | (R) | —CH₂CH₂CH₂— | | (S) | MeO— |
| | | | | H— | | | H— | |
| 58 | (CH₃)₃COOC— | H— | CH₃CH₂(CH₃)CH— | (R) | —CH₂CH₂CH₂— | | (S) | MeO— |
| | | | | H— | | | H— | |
| 59 | (CH₃)₃COOC— | H— | C₆H₅—CH₂— | (R) | —CH₂CH₂CH₂— | | (S) | MeO— |
| | | | | H— | | | H— | |
| 60 | (CH₃)₃COOC— | H— | HOCH₂— | (R) | —CH₂CH₂CH₂— | | (S) | MeO— |
| | | | | H— | | | H— | |
| 61 | (CH₃)₃COOC— | H— | (C₆H₅)₃CSCH₂— | (R) | —CH₂CH₂CH₂— | | (S) | MeO— |
| | | | | H— | | | H— | |
| 62 | (CH₃)₃COOC— | H— | CH₃CH(OH)— | (R) | —CH₂CH₂CH₂— | | (S) | MeO— |
| | | | | H— | | | H— | |
| 63 | (CH₃)₃COOC— | H— | CH₃SCH₂CH₂— | (R) | —CH₂CH₂CH₂— | | (S) | MeO— |
| | | | | H— | | | H— | |
| 64 | (CH₃)₃COOC— | H— | HOCH₂— | (R) | —CH₂CH₂CH₂— | | (R) | MeO— |
| | | | | H— | | | H— | |
| 65 | (CH₃)₃COOC— | H— | C₆H₅—CH₂— | (R) | —CH₂CH₂CH₂— | | (R) | MeO— |
| | | | | H— | | | H— | |
| 66 | (CH₃)₃COOC— | —CH₂CH₂CH₂— | | (R) | —CH₂CH₂CH₂— | | (R) | MeO— |
| | | | | H— | | | H— | |
| 67 | (CH₃)₃COOC— | H— | CH₃CH(OH)— | (R) | —CH₂CH₂CH₂— | | (R) | MeO— |
| | | | | H— | | | H— | |
| 68 | (CH₃)₃COOC— | H— | CH₃SCH₂CH₂— | (R) | —CH₂CH₂CH₂— | | (R) | MeO— |
| | | | | H— | | | H— | |
| 69 | (CH₃)₃COOC— | H— | CH₃CH₂(CH₃)CH— | (R) | —CH₂CH₂CH₂— | | (R) | MeO— |
| | | | | H— | | | H— | |
| 70 | (CH₃)₃COOC— | H— | CH₃— | (R) | —CH₂CH₂CH₂— | | (R) | MeO— |
| | | | | H— | | | H— | |
| 71 | (CH₃)₃COOC— | H— | (4-HO—C₆H₄)— | (R) | —CH₂CH₂CH₂— | | (R) | MeO— |
| | | | CH₂— | H— | | | H— | |
| 72 | (CH₃)₃COOC— | H— | NH₂COCH₂CH₂— | (R) | —CH₂CH₂CH₂— | | (R) | MeO— |
| | | | | H— | | | H— | |
| 73 | (CH₃)₃COOC— | H— | Indole-3-yl-CH₂— | (R) | —CH₂CH₂CH₂— | | (R) | MeO— |
| | | | | H— | | | H— | |
| 74 | (CH₃)₃COOC— | H— | (C₆H₅)₃CSCH₂— | (R) | —CH₂CH₂CH₂— | | (R) | MeO— |
| | | | | H— | | | H— | |
| 75 | (CH₃)₃COOC— | H— | (CH₃)₂CHCH₂— | (R) | —CH₂CH₂CH₂— | | (R) | MeO— |
| | | | | H— | | | H— | |
| 76 | (CH₃)₃COOC— | H— | (CH₃)₂CH— | (R) | —CH₂CH₂CH₂— | | (R) | MeO— |
| | | | | H— | | | H— | |
| 77 | (CH₃)₃COOC— | H— | (C₆H₅)₃CSCH₂— | (S) | —CH₂CH₂CH₂— | | (S) | MeO— |
| | | | | H— | | | H— | |
| 78 | H | H— | HSCH₂— | (S) | —CH₂CH₂CH₂— | | (S) | MeO— |
| | | | | H— | | | H— | |
| Com- | | | | | | | | |
| pound | | | | | | | | |
| No. | R¹ | R² | R³ | R⁴ | R⁵ | R⁶ | R⁷ | R⁸ |
| 79 | (CH₃)₃COOC— | H— | (CH₃)₂CHCH₂— | (S) | H— | CH₃CH(OH)— | (S) | MeO— |
| | | | | H— | | | H— | |
| 80 | (CH₃)₃COOC— | H— | CH₃CH₂(CH₃)CH— | (S) | H— | CH₃— | (S) | EtO— |
| | | | | H— | | | H— | |
| 81 | (CH₃)₃COOC— | H— | HOCH₂— | (S) | H— | H— | H— | MeO— |
| | | | | H— | | | | |
| 82 | (CH₃)₃COOC— | H— | H— | H— | H— | CH₃— | (S) | EtO— |
| | | | | | | | H— | |
| 83 | (CH₃)₃COOC— | H— | (CH₃)₂CHCH₂— | (S) | H— | C₆H₅—CH₂— | (S) | EtO— |
| | | | | H— | | | H— | |
| 84 | (CH₃)₃COOC— | H— | Indole-3-yl- | (S) | —CH₂CH₂CH₂— | | (S) | MeO— |
| | | | CH₂— | H— | | | H— | |
| 85 | (CH₃)₃COOC— | H— | CH₃CH₂(CH₃)CH— | (S) | —CH₂CH₂CH₂— | | (S) | MeO— |
| | | | | H— | | | H— | |
| 86 | (CH₃)₃COOC— | H— | CH₃— | (S) | —CH₂CH₂CH₂— | | (S) | MeO— |
| | | | | H— | | | H— | |
| 87 | (CH₃)₃COOC— | H— | H— | H— | H— | (CH₃)₂CH— | (S) | MeO— |
| | | | | | | | H— | |
| 88 | H— | H— | H— | H— | H— | (CH₃)₂CH— | (S) | MeO— |
| | | | | | | | H— | |
| 89 | H— | H— | HOOCCH₂— | (S) | H— | PhCH₂— | (S) | MeO— |
| | | | | H— | | | H— | |
| 90 | (CH₃)₃COOC— | H— | CH₃OOCCH₂— | (S) | H— | PhCH₂— | (S) | MeO— |
| | | | | H— | | | H— | |
| 91 | H— | H— | CH₃OOCCH₂— | (S) | H— | PhCH₂— | (S) | MeO— |
| | | | | H— | | | H— | |
| 92 | (CH₃)₃COOC— | H— | CH₂CHCH₂OOCCH₂— | (S) | H— | PhCH₂— | (S) | MeO— |
| | | | | H— | | | H— | |
| 93 | H— | H— | CH₂CHCH₂OOCCH₂— | (S) | H— | PhCH₂— | (S) | MeO— |
| | | | | H— | | | H— | |
| 94 | (CH₃)₃COOC— | H— | PhCH₂OOCCH₂— | (S) | H— | PhCH₂— | (S) | MeO— |
| | | | | H— | | | H— | |
| 95 | H— | H— | PhCH₂OOCCH₂— | (S) | H— | PhCH₂— | (S) | MeO— |
| | | | | H— | | | H— | |
| 96 | CH₃CO— | H— | CH₃OOCCH₂— | (S) | H— | PhCH₂— | (S) | MeO— |
| | | | | H— | | | H— | |
| 97 | PhCO— | H— | CH₃OOCCH₂— | (S) | H— | PhCH₂— | (S) | MeO— |
| | | | | H— | | | H— | |
| 98 | CH₃CO— | H— | CH₂CHCH₂OOCCH₂— | (S) | H— | PhCH₂— | (S) | MeO— |
| | | | | H— | | | H— | |
| 99 | PhCO— | H— | CH₂CHCH₂OOCCH₂— | (S) | H— | PhCH₂— | (S) | MeO— |
| | | | | H— | | | H— | |
| 100 | CH₃CO— | H— | PhCH₂OOCCH₂— | (S) | H— | PhCH₂— | (S) | MeO— |
| | | | | H— | | | H— | |
| 101 | PhCO— | H— | PhCH₂OOCCH₂— | (S) | H— | PhCH₂— | (S) | MeO— |
| | | | | H— | | | H— | |
| 102 | CH₃CO— | H— | HOOCCH₂— | (S) | H— | PhCH₂— | (S) | MeO— |
| | | | | H— | | | H— | |
| 103 | CH₃CO— | H— | (CH₃)₂CH— | (S) | H— | H— | H— | MeO— |
| | | | | H— | | | | |
| 104 | CH₃CO— | H— | (CH₃)₂CHCH₂— | (S) | H— | H— | H— | MeO— |
| | | | | H— | | | | |
| 105 | CH₃CO— | —CH₂CH₂CH₂— | | (S) | H— | H— | H— | MeO— |
| | | | | H— | | | | |
| 106 | (CH₃)₃COOC— | H— | H— | H— | —CH₂CH₂CH₂— | | (S) | MeO— |
| | | | | | | | H— | |
| 107 | H— | H— | Indole-3-yl- | (S) | —CH₂CH₂CH₂— | | (S) | MeO— |
| | | | CH₂— | H— | | | H— | |
| 108 | H— | H— | (CH₃)₂CH— | (S) | H— | CH₃— | (S) | MeO— |
| | | | | H— | | | H— | |
| 109 | H— | H— | CH₃CH₂(CH₃)CH— | (S) | H— | CH₃— | (S) | MeO— |
| | | | | H— | | | H— | |

Compound 1

Colorless liquid; ¹H NMR (400 MHz, CD₃OD) δ 4.17 (1H, t, J=4 Hz), 4.50 (1H, dd, J=6.0, 9.0 Hz), 3.75 (2H, dd, J=11.2, 5.2 Hz), 3.70 (3H, s), 1.63 (1H, m), 1.70-1.62 (2H, m), 1.45 (9H, br s), 0.93 (3H, d, J=6.8 Hz), 0.92 (3H, d, J=6.8 Hz).

Compound 2

Colorless liquid; ¹H NMR (400 MHz, CD₃OD) δ 4.51 (1H, m), 4.50 (1H, m), 3.87 (2H, d, J=11.2, 5.2 Hz), 3.70 (3H, s), 1.66-1.63 (2H, m), 1.62 (1H, m), 0.95 (3H, d, J=6.4 Hz), 0.92 (3H, d, J=6.4 Hz).

Compound 3

White crystal; ¹H NMR (400 MHz, CD₃OD) δ 4.12 (1H, m), 3.97 (1H, d, J=17.6 Hz), 3.88 (1H, d, J=17.6 Hz), 3.70 (3H, s), 1.71 (2H, m), 1.53 (1H, m), 1.44 (9H, br s), 0.95 (3H, d, J=6.8 Hz), 0.93 (3H, d, J=6.8 Hz); ¹³C NMR (100 MHz, CD₃OD) δ 174.9, 170.1, 156.4, 79.1, 52.9, 51.1, 40.8, 40.4, 27.2, 24.4, 22.0, 20.4.

Compound 4

¹H NMR (400 MHz, CD₃OD) δ 4.07 (2H, d, J=17.6 Hz), 3.90 (1H, m), 3.72 (3H, s), 1.63 (1H, m), 1.60-1.70 (2H, m), 0.93 (3H, d, J=6.4 Hz), 0.90 (3H, d, J=6.4 Hz).

Compound 5

White solid; ¹H NMR (400 MHz, CD₃OD) δ 0.93 (3H, d, J=6.8 Hz), 0.97 (3H, d, J=7.2 Hz), 1.44 (9H, br s), 2.06 (1H, m), 3.67 (3H, s), 3.91 (1H, m), 3.97 (2H, m); ¹³C NMR (100 MHz, CD₃OD) δ 173.6, 170.0, 156.5, 79.1, 59.9, 51.1, 40.3, 30.6, 27.2, 18.2, 16.8.

Compound 6

White solid; ¹H NMR (400 MHz, CD₃OD) δ 1.06 (3H, d, J=2.8 Hz), 1.08 (3H, d, J=2.8 Hz), 2.20 (1H, m), 3.71 (3H, s), 3.74 (1H, d, J=5.6 Hz), 3.93 (1H, d, J=17.6 Hz), 4.09 (1H, d, J=17.6 Hz); ¹³C NMR (100 MHz, CD₃OD) δ 171.5, 170.3, 59.8, 52.8, 41.9, 31.6, 18.7, 18.0.

Compound 7

Colorless liquid; ¹H NMR (400 MHz, CD₃OD) δ 4.50 (1H, dd, J=6.0, 5.6 Hz), 4.05 (1H, m), 4.00 (1H, m), 3.69 (3H, s), 1.70 (1H, m), 1.68 (1H, m), 1.60 (1H, m), 1.44 (9H, br s), 1.19 (3H, d, J=6.4 Hz), 0.94 (3H, d, J=6.4 Hz), 0.91 (3H, d, J=6.4 Hz); ³C NMR (100 MHz, CD₃OD) δ 173.0, 172.0, 156.5, 79.3, 67.1, 59.9, 51.2, 50.6, 40.1, 27.2, 24.3, 21.8, 20.4, 18.6.

Compound 8

White solid; ¹H NMR (400 MHz, CD₃OD) δ 4.47 (1H, dd, J=6.0, 6.0 Hz), 4.35 (1H, d, J=6 Hz), 4.10 (1H, q. J=6 Hz), 3.70 (3H, s), 1.70 (1H, m), 1.68 (1H, m), 1.60 (1H, m), 1.21 (3H, d, J=6.4 Hz), 0.95 (3H, d, J=6.8 Hz), 0.90 (3H, d, J=6.8 Hz).

Compound 9

White solid; ¹H NMR (400 MHz, CD₃OD) δ 4.38 (1H, q, J=7.6 Hz), 4.16 (2H, q, J=7.2 Hz), 4.11 (1H, t, J=4.4 Hz), 1.73 (2H, m), 1.52 (1H, m), 1.43 (9H, br s), 1.38 (3H, d, J=7.2 Hz), 1.25 (3H, t, J=7.2 Hz), 0.95 (3H, d, J=6.8 Hz), 0.93 (3H, d, J=6.8 Hz).

Compound 10

White solid; ¹H NMR (400 MHz, CD₃OD) δ 4.38 (1H, q, J=7.0 Hz), 4.16 (2H, q, J=7.2 Hz), 3.36 (1H, t, J=6.4 Hz), 1.73 (2H, m), 1.52 (1H, m), 1.38 (3H, d, J=7.2 Hz), 1.25 (3H, t, J=7.2 Hz), 0.96 (3H, d, J=6.4 Hz), 0.93 (3H, d, J=6.4 Hz); ¹³C NMR (100 MHz, CD₃OD) δ 176.3, 172.6, 160.8, 52.7, 48.2, 44.2, 24.2, 21.8, 21.2, 16.0, 13.0.

Compound 11

White solid; ¹H NMR (400 MHz, CD₃OD) δ 1.84 (1H, m), 1.61 (1H, dd, J=8.4, 5.6 Hz), 1.64 (1H, dd, J=8.4, 5.2 Hz), 3.93 (1H, ddd, J=8.4, 4.4, 0.8 Hz), 3.99 (1H, ddd, J=14.0, 6.8, 0.8 Hz), 1.44 (3H, d, J=6.8 Hz), 0.96 (3H, t, J=6.8 Hz), 0.96 (3H, t, J=6.8 Hz).

Compound 12

White solid; ¹H NMR (CDCl₃, 400 MHz) δ 8.10 (1H, s), 7.96 (1H, s), 4.60 (1H, q), 4.52 (1H, d), 3.67 (3H, s), 2.66 (1H, m), 1.48 (3H, s), 1.40 (9H, s), 1.01 (6H, d); ¹³C NMR (CDCl₃, 100 MHz) δ 171.6, 171.1, 156.0, 79.5, 60.3, 51.9, 48.2, 31.0, 28.5, 17.1.

Compound 13

White solid; ¹H NMR (CDCl₃, 400 MHz) δ 6.68 (1H, s), 5.12 (1H, s), 4.59-4.53 (1H, m), 4.17 (1H, s), 3.73 (3H, s), 1.43 (9H, s), 1.37-1.31 (6H, m); ¹³C NMR (CDCl₃, 100 MHz) δ 173.1, 172.3, 155.4, 80.0, 52.4, 49.9, 47.9, 28.2, 18.3, 18.2.

Compound 14

Viscous liquid; ¹H NMR (400 MHz, CDCl₃): δ 6.50 (1H, s), 6.22 (1H, s), 4.74-4.69 (1H, m), 3.59 (3H, s), 3.49-3.47 (2H, d), 1.54 (9H, s), 1.29-1.27 (3H, d); ¹³C NMR (CDCl₃, 100 MHz) δ 171.6, 170.7, 156.3, 79.5, 51.9, 47.9, 45.0, 28.5, 17.2.

Compound 15

Viscous liquid; ¹H NMR (400 MHz, CDCl₃) δ 7.02 (2H, d), 6.74 (2H, d), 6.55 (1H, d), 5.11 (1H, s), 4.52 (1H, t), 4.29 (1H, s), 3.70 (3H, s), 2.97 (2H, dd), 1.41 (9H, s), 1.33 (3H, d); ¹³C NMR (CDCl₃, 100 MHz) δ 171.6, 170.7, 156.3, 79.5, 51.9, 47.9, 45.0, 28.5, 17.2.

Compound 16

White solid; ¹H NMR (CDCl₃, 400 MHz) δ 8.34 (1H, s), 7.50 (1H, d), 7.34 (1H, d), 7.19 (1H, t), 7.12 (1H, t), 6.97 (1H, d), 6.63 (1H, d), 5.11 (1H, m), 4.93 (1H, m), 3.73 (2H, s), 3.66 (3H, s), 3.31 (2H, d), 1.41 (9H, s); ¹³C NMR (CDCl₃, 100 MHz) δ 172.1, 169.1, 156.2, 136.1, 127.5, 123.0, 122.2, 119.6, 118.3, 111.3, 109.6, 80.2, 52.8, 52.4, 44.2, 38.6, 28.2, 27.5.

Compound 17

White solid; ¹H NMR (DMSO-d₆, 400 MHz) δ 8.95 (1H, s), 7.50-7.48 (1H, d), 7.16-7.02 (4H, m), 6.72 (1H, s), 6.02 (1H, s), 4.88-4.84 (1H, m), 4.28-4.23 (1H, m), 3.54 (3H, s), 3.25-3.23 (2H, d), 1.55 (9H, s), 1.52-1.47 (1H, m), 1.01-0.99 (6H, d); ¹³C NMR (DMSO-d₆, 400 MHz) δ 175.8, 171.2, 152.5, 135.9, 128.5, 122.8, 120.7, 119.6, 118.1, 112.2, 109.3, 79.9, 55.6, 51.9, 37.8, 28.7 (3C), 26.6, 24.2 (2C), 21.4.

Compound 18

Viscous liquid; ¹H NMR (CDCl₃, 400 MHz) δ 7.2 (1H, s), 5.29 (1H, s), 4.45 (2H, d), 3.85 (2H, d), 3.67 (3H, s), 1.45 (9H, s); ¹³C NMR (CDCl₃, 100 MHz) δ 171.5, 171.0, 155.5, 80.3, 51.2, 48.5, 46.1, 29.0.

Compound 19 ¹H NMR (CDCl₃, 400 MHz) δ 7.31-7.20 (5H, m), 5.24 (1H, d), 4.67 (1H, dd), 4.50 (1H, dd), 3.74 (3H, s), 3.61-3.57 (1H, m), 3.19-3.15 (1H, m), 3.11 (1H, dd), 2.93 (1H, dd), 2.17-2.13 (1H, m), 1.96-1.88 (3H, m), 1.37 (9H, s); ¹³C NMR (CDCl₃, 100 MHz) δ 172.5, 170.8, 155.3, 136.5, 129.9, 128.5, 126.9, 79.8, 60.6, 59.1, 53.4, 52.4, 47.0, 39.4, 29.2, 28.5, 25.0.

Compound 20

Viscous liquid; ¹H NMR (CD₃OD, 400 MHz) δ 4.46 (1H, dd, J=4.8, 8.8 Hz), 4.37 (1H, q, J=6.8 Hz), 3.77 (1H, m), 3.69 (3H, s), 3.65-3.62 (1H, m), 3.60-3.54 (1H, m), 2.30-2.23 (1H, m), 2.07-1.93 (3H, m), 1.42 (9H, s), 1.28 (3H, d, J=7.6 Hz); ¹³C NMR (CDCl₃, 100 MHz) δ 172.7, 172.6, 156.2, 79.0, 58.9, 51.3, 47.7, 46.6, 28.5, 27.3, 24.5, 15.7.

Compound 21

¹H NMR (CDCl₃, 400 MHz) δ 5.19 (1H, d), 4.52 (1H, dd), 4.28 (1H, dd), 3.77-3.73 (1H, m), 3.70 (3H, s), 3.64-3.62 (1H, m), 3.59-3.56 (1H, m), 2.23-2.19 (1H, m), 2.03-1.94 (3H, m), 1.40 (9H, s), 1.02 (3H, d), 0.92 (3H, d); ¹³C NMR (CDCl₃, 100 MHz) δ 172.6, 171.4, 156.0, 79.6, 58.9, 57.0, 52.3, 47.3, 31.5, 29.2, 28.5, 25.2, 19.4, 17.5.

Compound 22

¹H NMR (CDCl₃, 400 MHz) δ 5.09 (1H, d), 4.45 (1H, dd), 4.39 (1H, dd), 3.71-3.68 (1H, m), 3.62 (3H, s), 3.53-3.51 (1H, m), 2.16-2.11 (1H, m), 2.01-1.86 (3H, m), 1.72-1.65 (1H. m), 1.42 (1H, t), 1.33 (9H, s), 0.92 (3H, d), 0.87 (3H, d); ¹³C NMR (CDCl₃, 100 MHz) δ 172.6, 171.9, 155.8, 79.5, 58.8, 52.3, 50.4, 46.8, 42.0, 29.1, 28.4, 25.0, 24.6, 23.5, 21.9.

Compound 23

White solid; ¹H NMR (CDCl₃, 400 MHz) δ 6.50 (1H, s), 5.95 (1H, s), 5.57 (1H, d), 4.47-4.42 (2H, m), 3.66-3.63 (2H, t), 3.64 (3H, s), 2.27-2.22 (2H, m), 2.16-2.04 (2H, m), 1.97-1.89 (3H, m), 1.81-1.77 (1H, m), 1.35 (9H, s); ¹³C NMR (CDCl₃, 100 MHz) δ 175.1, 172.5, 170.6, 155.8, 79.7, 58.7, 52.2, 50.9, 46.9, 31.2, 29.0, 28.9, 28.3, 28.2, 24.9.

Compound 24

Viscous liquid; ¹H NMR (CDCl₃, 400 MHz) δ 5.48 (1H, d), 4.50 (1H, dd), 4.38 (1H, dd), 4.14 (1H, m), 3.76-3.74 (1H, m), 3.69 (3H, s), 3.47 (1H, s), 2.22-2.20 (1H, m), 2.01-1.93 (3H, m), 1.40 (9H, s), 1.19 (3H, d); ¹³C NMR (CDCl₃, 100 MHz) δ 172.5, 170.9, 156.1, 79.9, 67.4, 58.8, 55.7, 52.7, 52.4, 47.2, 38.5, 31.3, 28.9, 24.8, 18.5.

Compound 25

Viscous liquid; ¹H NMR (CDCl₃, 400 MHz) δ 5.32 (1H, d), 4.81 (1H, dd), 4.46 (1H, dd), 3.73-3.69 (2H, m), 3.67 (3H, s), 2.65 (1H, dd), 2.45 (1H, dd), 2.17-2.12 (1H, m), 2.01-1.90 (3H, m), 1.39 (9H, s), 1.35 (9H, s); ¹³C NMR (CDCl₃, 100 MHz) δ 172.4, 172.2, 170.0, 155.0, 80.9, 79.7, 59.3, 58.8, 53.4, 52.5, 52.1, 49.1, 48.9, 46.8, 46.5, 31.2, 28.9, 27.9, 24.7.

Compound 26

Viscous liquid; ¹H NMR (CDCl₃, 400 MHz) δ 6.85 (1H, s), 6.17 (1H, d), 6.02 (1H, s), 4.78 (1H, dd), 4.42 (1H, dd), 3.76-3.70 (1H, m), 3.61 (3H, s), 2.64-2.46 (1H, m), 2.17-2.12 (1H, m), 2.16-2.04 (2H, m), 1.99-1.89 (3H, m), 1.34 (9H, s); ¹³C NMR (CDCl₃, 100 MHz) δ 172.8, 172.4, 171.0, 155.5, 79.7, 59.1, 52.5, 52.2, 49.4, 47.1, 46.6, 37.8, 28.9, 28.3, 24.8.

Compound 27

Viscous liquid; ¹H NMR (CDCl₃, 400 MHz) δ 4.50 (1H, dd), 4.43 (1H, dd), 3.69 (3H, s), 3.57-3.28 (4H, m), 2.12-1.73 (8H, m), 1.37 (9H, s); ¹³C NMR (CDCl₃, 100 MHz) δ 172.7, 171.6, 157.7, 79.8, 60.5, 58.7, 51.9, 47.1, 46.0, 29.7, 28.5, 22.6, 22.1.

Compound 28

Viscous liquid; ¹H NMR (CDCl₃, 400 MHz) δ 5.30 (1H, d), 4.83 (1H, s), 4.54 (1H, dd), 4.46 (1H, dd), 3.72 (3H, s), 3.63-3.58 (1H, m), 3.13-3.08 (2H, m), 2.24-2.18 (2H, m), 2.07-1.93 (3H, m), 1.76-1.73 (1H, m), 1.60-1.37 (5H, m), 1.42 (18H, s); ¹³C NMR (CDCl₃, 100 MHz) δ 172.5, 171.6, 156.0, 155.2, 79.6, 58.7, 52.3, 51.5, 46.9, 32.3, 28.9, 28.4, 28.3, 24.9, 21.9.

Compound 29

White solid; ¹H NMR (CDCl₃, 400 MHz) δ 7.67 (1H, s), 7.42 (1H, s), 6.90 (1H, s), 5.59 (1H, d), 4.59 (1H, dd), 4.48 (1H, dd), 3.71 (3H, s), 3.62-3.58 (1H, m), 3.27-3.22 (1H, m), 3.10-2.97 (2H, m), 2.19 (1H, dd), 1.98-1.82 (3H, m), 1.35 (9H, s); ¹³C NMR (CDCl₃, 100 MHz) δ 173.4, 170.6, 155.2, 135.2, 127.9, 122.5, 79.9, 59.0, 52.6, 51.9, 47.0, 29.5, 28.9, 28.8, 28.3, 28.2, 25.1.

Compound 30

White solid; ¹H NMR (CDCl₃, 400 MHz) δ 5.34 (1H, d), 4.54-4.49 (1H, m), 4.39 (1H, dd), 3.69 (3H, s), 3.66-3.54 (2H, m), 2.34-2.18 (2H, m), 2.17-1.87 (5H, m), 1.76-1.67 (1H, m), 1.41 (18H, s); ¹³C NMR (CDCl₃, 100 MHz) δ 172.3, 172.1, 170.5, 155.5, 80.4, 79.5, 59.2, 52.6, 52.2, 51.3, 51.0, 49.1, 48.9, 46.9, 46.4, 31.4, 30.6, 28.0, 27.8, 24.7.

Compound 31

Light yellow solid; ¹H NMR (CDCl₃, 400 MHz) δ 9.38 (1H, s), 9.18 (1H, s), 7.29-7.16 (10H, m), 5.32 (1H, d), 5.14 (2H, s), 5.03 (2H, s), 4.38 (1H, dd), 4.34 (1H, dd), 3.92 (2H, m), 3.56-3.52 (1H, m), 3.50 (3H, s), 3.42-3.38 (1H, m), 2.67 (2H, d), 2.06-2.00 (1H, m), 1.82-1.74 (2H, m), 1.39-1.21 (3H, m), 1.31 (9H, s); ¹³C NMR (CDCl₃, 100 MHz) δ 172.1, 170.8, 165.5, 163.7, 160.5, 160.4, 155.7, 155.7, 155.4, 137.0, 134.8, 128.6, 128.6, 128.3, 127.9, 127.8, 127.8, 127.6, 79.3, 68.7, 66.8, 58.7, 51.9, 51.5, 46.7, 44.3, 38.5, 29.7, 28.8, 28.3, 24.8, 24.5

Compound 32

White solid; ¹H NMR (CDCl₃, 400 MHz) δ 7.57 (1H, s), 7.05 (2H, d), 6.70 (2H, d), 5.30 (1H, d), 4.61 (1H, dd), 4.49 (1H, dd), 3.68 (3H, s), 3.61-3.59 (1H, m), 3.28-3.26 (1H, m), 3.00 (1H, dd), 2.82 (1H, dd), 2.13-2.09 (1H, m), 1.95-1.88 (3H, m), 1.35 (9H, s); ¹³C NMR (CDCl₃, 100 MHz) δ 172.5, 171.2, 155.8, 130.9, 127.1, 115.6, 80.2, 60.6, 59.2, 53.5, 52.4, 47.1, 38.0, 29.1, 28.5, 25.0, 21.2, 14.3.

Compound 33

White solid; ¹H NMR (CDCl₃, 400 MHz) δ 4.44 (1H, dd), 4.39 (1H, dd), 3.69 (3H, s), 3.61-3.33 (4H, m), 2.21-1.78 (8H, m), 1.43 (9H, s); ¹³C NMR (CDCl₃, 100 MHz) δ 173.2, 172.6, 153.9, 79.9, 59.5, 58.0, 52.3, 52.0, 47.1, 46.0, 30.0, 29.4, 28.4, 24.5.

Compound 34

White solid; ¹H NMR (CDCl₃, 400 MHz) δ 5.16 (1H, d), 4.42 (1H, dd), 4.34 (1H, dd), 3.90-3.86 (1H, m), 3.70 (3H, s), 3.57-3.51 (1H, m), 2.20-2.17 (1H, m), 2.04-1.92 (3H, m), 1.71-1.66 (1H, m), 1.59-1.53 (1H, m), 1.41 (9H, s), 1.16-1.10 (1H, m), 0.91 (3H, d), 0.88 (3H, t); ¹³C NMR (CDCl₃, 100 MHz) δ 172.3, 171.1, 155.7, 79.4, 59.1, 56.2, 52.3, 52.1, 47.1, 37.9, 31.1, 29.1, 28.3, 24.6, 24.1, 22.3, 15.6, 11.3, 10.9.

Compound 35

White solid; ¹H NMR (CDCl₃, 400 MHz) δ 7.44 (1H, s), 7.00 (2H, d), 6.72 (2H, d), 5.43 (1H, d), 4.59 (1H, dd), 4.29 (1H, dd), 3.68 (3H, s), 3.55-3.49 (1H, m), 2.93 (1H, dd), 2.84 (1H, dd), 2.73-2.69 (1H, m), 2.13-2.09 (1H, m), 1.94-1.78 (3H, m), 1.36 (9H, s); ¹³C NMR (CDCl₃, 100 MHz) δ 172.3, 170.7, 155.66, 130.4, 127.8, 115.4, 80.0, 60.5, 59.4, 58.8, 53.7, 52.6, 46.9, 39.2, 38.6, 31.1, 28.3, 24.4, 21.2.

Compound 36

Viscous liquid; ¹H NMR (CDCl₃, 400 MHz) δ 5.20 (1H, d), 4.48 (1H, dd), 4.38 (1H, dd), 3.85-3.80 (1H, m), 3.67 (3H, s), 3.54-3.43 (2H, m), 2.17-2.02 (2H, m), 1.98-1.91 (2H, m), 1.68-1.65 (1H. m), 1.41 (1H, t), 1.38 (9H, s), 0.94 (3H, d), 0.88 (3H, d); ¹³C NMR (CDCl₃, 100 MHz) δ 172.6, 171.9, 155.8, 79.5, 59.1, 52.3, 50.4, 46.7, 42.0, 29.1, 28.2, 24.6, 24.2, 23.3, 21.9.

Compound 37

Viscous liquid; ¹H NMR (CDCl₃, 400 MHz) δ 5.22 (1H, d), 4.78 (1H, dd), 4.41 (1H, dd), 3.80-3.76 (1H, m), 3.67 (3H, s), 3.66-3.54 (1H, m), 2.73-2.67 (1H, dd), 2.50 (1H, dd), 2.19-2.14 (1H, m), 2.05-1.90 (3H, m), 1.35 (18H, s); ¹³C NMR (CDCl₃, 100 MHz) δ 172.3, 169.7, 169.5, 154.9, 80.9, 79.8, 59.1, 59.0, 52.6, 52.0, 49.3, 48.9, 46.9, 46.6, 31.0, 29.0, 28.2, 27.9, 24.8, 22.4.

Compound 38

Viscous liquid; ¹H NMR (CDCl₃, 400 MHz) δ 5.39 (1H, s), 4.50 (1H, dd), 3.94 (1H, dd), 3.69 (3H, s), 3.59-3.55 (1H, m), 3.46-3.40 (1H, m), 2.21-2.14 (1H, m), 2.10-1.94 (4H, m), 1.40 (9H, s); ¹³C NMR (CDCl₃, 100 MHz) δ 172.8, 167.7, 156.2, 80.0, 58.8, 52.7, 46.2, 43.4, 29.4, 28.7, 25.0.

Compound 39

Viscous liquid; ¹H NMR (CDCl₃, 400 MHz) δ 5.66 (1H, d), 4.55 (1H, dd), 4.46 (1H, dd), 4.38 (1H, m), 3.81-3.77 (1H, m), 3.71 (3H, s), 3.67-3.49 (2H, m), 3.37 (1H, s), 2.26-2.20 (1H, m), 2.11-1.99 (1H, m), 2.01-1.91 (2H, m), 1.40 (9H, s); ¹³C NMR (CDCl₃, 100 MHz) δ 172.6, 169.7, 155.8, 80.1, 63.8, 59.4, 53.5, 52.8, 52.3, 47.1, 30.8, 29.0, 24.7, 22.4.

Compound 40

Viscous liquid; ¹H NMR (CDCl₃, 400 MHz) δ 5.33 (1H, d), 4.57 (1H, dd), 4.36 (1H, dd), 3.79-3.76 (1H, m), 3.64 (3H, s), 3.55-3.48 (1H, m), 2.50-2.42 (2H, m), 2.17-2.12 (1H, m), 2.01 (3H, s), 1.99-1.83 (4H, m), 1.80-1.70 (1H, m), 1.35 (9H, s); ¹³C NMR (CDCl₃, 100 MHz) δ 172.3, 170.4, 155.3, 79.7, 59.1, 53.4, 51.0, 46.9, 32.6, 31.1, 30.1, 30.8, 24.6, 22.3, 15.5.

Compound 41

Viscous liquid; ¹H NMR (CDCl₃, 400 MHz) δ 5.52 (1H, d), 4.80 (1H, dd), 4.50 (1H, dd), 3.72 (3H, s), 3.67-3.62 (1H, m), 2.78 (2H, t), 2.28-2.20 (1H, m), 2.10-1.92 (4H, m), 1.81-1.77 (1H, m), 1.35 (9H, s), 0.86-0.81 (1H, m); ¹³C NMR (CDCl₃, 100 MHz) δ 172.3, 172.1, 167.3, 154.7, 80.8, 59.3, 53.0, 52.4, 48.8, 47.2, 46.8, 38.6, 29.0, 28.2, 24.7.

Compound 42

Viscous liquid; ¹H NMR (CDCl₃, 400 MHz) δ 7.23-7.15 (5H, m), 5.38 (1H, d), 4.61 (1H, dd), 4.25 (1H, dd), 3.66 (3H, s), 3.49-3.45 (1H, m), 3.02 (1H, dd), 2.90 (1H, dd), 2.62-2.57 (1H, m), 1.92-1.75 (3H, m), 1.49-1.43 (1H, m), 1.37 (9H, s); ¹³C NMR (CDCl₃, 100 MHz) δ 172.3, 170.2, 154.9, 136.4, 129.4, 128.3, 126.8, 79.6, 58.6, 53.5, 52.5, 46.7, 40.3, 28.9, 28.3, 24.3, 22.4.

Compound 43

White solid; ¹H NMR (CDCl₃, 400 MHz) δ 8.45 (1H, s), 7.58 (1H, d), 7.32 (1H, d), 7.16 (1H, t), 7.09 (1H, t), 7.05 (1H, d), 5.52 (1H, d), 4.70-4.64 (1H, m), 4.15 (1H, dd), 3.67 (3H, s), 3.40-3.34 (1H, m), 3.27 (1H, dd), 3.15 (1H, dd), 2.47-2.41 (1H, m), 1.67-1.44 (3H, m), 1.44 (9H, s), 1.10-1.06 (1H, m); ¹³C NMR (CDCl₃, 100 MHz) δ 172.5, 170.9, 155.1, 136.0, 127.5, 123.1, 121.8, 119.4, 118.6, 111.1, 110.7, 79.6, 58.9, 53.4, 52.3, 46.6, 31.4, 30.0, 29.7, 28.7, 28.2, 24.1.

Compound 44

White solid; ¹H NMR (CDCl₃, 400 MHz) δ 5.44 (1H, d), 4.48 (1H, q), 4.45 (1H, dd), 3.79-3.74 (1H, m), 3.69 (3H, s), 3.51-3.44 (1H, m), 2.20-2.16 (1H, m), 2.14-1.92 (3H, m), 1.38 (9H, s), 1.30 (3H, d); ¹³C NMR (CDCl₃, 100 MHz) δ 175.5, 172.9, 155.4, 79.5, 49.5, 46.2, 36.9, 35.8, 29.4, 28.3, 18.5.

Compound 45

White solid; ¹H NMR (CDCl₃, 400 MHz) δ 6.62 (1H, s), 5.82 (1H, s), 5.69 (1H, d), 4.45 (1H, dd), 4.40 (1H, dd), 3.69 (3H, s), 3.59-3.55 (1H, m), 2.31-2.16 (5H, m), 2.05-1.93 (3H, m), 1.79-1.75 (1H, m), 1.40 (9H, s); ¹³C NMR (CDCl₃, 100 MHz) δ 175.4, 172.5, 170.4, 156.1, 80.0, 59.4, 52.3, 51.2, 46.8, 31.5, 29.5, 29.0, 28.2, 24.6, 22.2.

Compound 46

White solid; ¹H NMR (CDCl₃, 400 MHz) δ 7.49 (1H, s), 7.42 (1H, s), 6.78 (1H, s), 5.63 (1H, d), 4.68 (1H, dd), 4.31 (1H, dd), 3.71 (3H, s), 3.63-3.49 (2H, m), 3.20-3.14 (1H, m), 2.95-2.86 (1H, m), 2.05-2.01 (1H, m), 1.98-1.70 (3H, m), 1.34 (9H, s); ¹³C NMR (CDCl₃, 100 MHz) δ 172.3, 170.8, 155.2, 135.1, 131.1, 119.6, 79.8, 59.3, 53.4, 52.3, 46.9, 30.3, 28.9, 28.2, 24.6, 22.3.

Compound 47

Viscous liquid; ¹H NMR (CDCl₃, 400 MHz) δ 5.50 (1H, d), 5.10 (1H, dd), 4.43 (1H, dd), 4.35 (1H, dd), 4.15 (1H, q), 3.83-3.78 (1H, m), 3.69 (3H, s), 3.66-3.64 (1H, m), 2.20-2.17 (1H, m), 2.04-1.92 (3H, m), 1.43 (9H, s), 1.15 (3H, d); ¹³C NMR (CDCl3, 100 MHz) δ 172.5, 171.0, 156.1, 80.0, 67.5, 59.0, 55.1, 54.0, 52.7, 52.3, 47.2, 38.6, 30.7, 29.0, 24.8, 18.7.

Compound 48

Viscous liquid; ¹H NMR (CDCl₃, 400 MHz) δ 5.33 (1H, d), 4.88 (1H, s), 4.53 (1H, dd), 4.43 (1H, dd), 3.70 (3H, s), 3.61-3.59 (1H, m), 3.11-3.08 (2H, m), 2.21-2.14 (2H, m), 2.07-1.91 (3H, m), 1.76-1.72 (1H, m), 1.61-1.38 (5H, m), 1.40 (18H, s); ¹³C NMR (CDCl₃, 100 MHz) δ 172.4, 171.1, 156.0, 155.5, 79.5, 78.8, 58.6, 52.2, 51.5, 46.8, 40.1, 32.2, 29.3, 28.9, 28.4, 28.3, 24.9, 21.9.

Compound 49

Viscous liquid; ¹H NMR (CDCl₃, 400 MHz) δ 5.10 (1H, d), 4.22 (1H, dd), 4.12 (1H, dd), 3.72-3.764 (1H, m), 3.51 (3H, s), 3.41-3.33 (1H, m), 2.03-1.72 (5H, m), 1.24 (9H, s), 0.77 (3H, d), 0.72 (3H, d); ¹³C NMR (CDCl₃, 100 MHz) δ 172.2, 170.7, 155.5, 79.0, 58.7, 56.7, 51.9, 46.9, 31.1, 28.9, 28.1, 24.5, 19.5, 19.4, 17.2.

Compound 50

Viscous liquid; ¹H NMR (CDCl₃, 400 MHz) δ 5.23 (1H, d), 4.50-4.48 (2H, m), 3.72-3.65 (2H, m), 3.68 (3H, s), 2.38-2.14 (3H, m), 2.09-1.88 (4H, m), 1.76-1.67 (1H, m), 1.42 (18H, s); ¹³C NMR (CDCl₃, 100 MHz) δ 172.2, 172.1, 170.8, 155.5, 80.4, 79.5, 59.7, 52.1, 50.9, 46.8, 30.7, 28.9, 28.3, 28.0, 27.7, 24.9.

Compound 51

White solid; ¹H NMR (CDCl₃, 400 MHz) δ 5.44 (1H, d), 4.48 (1H, q), 4.45 (1H, dd), 3.79-3.74 (1H, m), 3.69 (3H, s), 3.50-3.44 (1H, m), 2.21-2.16 (1H, m), 2.12-1.92 (3H, m), 1.38 (9H, s), 1.29 (3H, d); ¹³C NMR (CDCl₃, 100 MHz) δ 172.6, 171.8, 155.2, 79.6, 59.0, 52.5, 47.9, 46.8, 29.0, 28.3, 24.6, 22.4, 18.7.

Compound 52

White solid; ¹H NMR (CDCl₃, 400 MHz) δ 8.68 (1H, s), 7.57 (1H, d), 7.32 (1H, d), 7.15 (1H, t), 7.08 (1H, t), 7.02 (1H, d), 5.54 (1H, d), 4.68-4.65 (1H, m), 4.14 (1H, dd), 3.65 (3H, s), 3.38-3.33 (1H, m), 3.26-3.21 (1H, m), 3.15 (1H, dd), 2.46-2.41 (1H, m), 1.65-1.51 (3H, m), 1.44 (9H, s), 1.10-1.06 (1H, m); ¹³C NMR (CDCl₃, 100 MHz) δ 172.5, 170.9, 155.1, 136.0, 127.5, 123.2, 121.8, 111.2, 79.6, 58.9, 53.4, 52.1, 46.6, 38.6, 30.0, 28.7, 24.1.

Compound 53 Light yellow solid; ¹H NMR (CDCl₃, 400 MHz) δ 7.60 (1H, s), 6.99 (2H, d), 6.72 (2H, d), 5.43 (1H, d), 4.57 (1H, dd), 4.28 (1H, dd), 3.66 (3H, s), 3.52-3.50 (1H, m), 2.93 (1H, dd), 2.83 (1H, dd), 2.72-2.66 (1H, m), 1.96-1.76 (3H, m), 1.56-1.48 (1H, m), 1.40 (9H, s); ¹³C NMR (CDCl₃, 100 MHz) δ 172.6, 170.7, 155.6, 130.4, 127.7, 115.4, 80.0, 58.9, 53.4, 52.6, 52.3, 46.9, 39.2, 28.9, 28.2, 24.4, 22.3.

Compound 54

White solid; ¹H NMR (CDCl₃, 400 MHz) δ 6.65 (1H, s), 5.63 (1H, dd), 5.55 (1H, s), 4.49 (1H, dd), 4.41 (1H, dd), 3.71 (3H, s), 3.68-3.55 (2H, m), 2.35-2.14 (3H, m), 2.10-1.95 (4H, m), 1.79-1.71 (1H, m), 1.43 (9H, s); ¹³C NMR (CDCl₃, 100 MHz) δ 175.0, 172.4, 170.2, 156.2, 79.9, 59.1, 52.2, 51.1, 46.7, 31.6, 30.0, 29.1, 28.3, 24.6, 22.2.

Compound 55

White solid; ¹H NMR (CDCl₃, 400 MHz) δ 4.41 (1H, dd), 4.35 (1H, dd), 3.64 (3H, s), 3.59-3.29 (4H, m), 2.17-1.74 (8H, m), 1.39 (9H, s); ¹³C NMR (CDCl₃, 100 MHz) δ 173.1, 172.4, 154.4, 79.8, 59.1, 58.0, 52.0, 46.5, 46.4, 31.5, 29.8, 28.9, 28.4, 24.9, 23.2, 22.5.

Compound 56

Viscous liquid; ¹H NMR (CDCl₃, 400 MHz) δ 5.20 (1H, d), 4.38 (1H, dd), 4.29 (1H, dd), 3.87-3.81 (1H, m), 3.67 (3H, s), 3.54-3.48 (1H, m), 2.17-1.87 (5H, m), 1.39 (9H, s), 0.92 (3H, d), 0.87 (3H, d); ¹³C NMR (CDCl₃, 100 MHz) δ 172.4, 170.9, 155.7, 79.3, 58.8, 56.8, 52.3, 47.0, 31.3, 29.1, 28.3, 24.6, 19.6, 17.3.

Compound 57

Viscous liquid; ¹H NMR (CDCl₃, 400 MHz) δ 5.20 (1H, d), 4.47 (1H, dd), 4.37 (1H, dd), 3.84-3.81 (1H, m), 3.65 (3H, s), 3.53-3.44 (2H, m), 2.23-2.02 (2H, m), 1.98-1.91 (2H, m), 1.68-1.62 (1H. m), 1.41 (1H, t), 1.37 (9H, s), 0.94 (3H, d), 0.87 (3H, d); ¹³C NMR (CDCl₃, 100 MHz) δ 172.3, 171.6, 155.4, 79.4, 58.9, 52.3, 52.1, 50.3, 46.7, 42.6, 29.0, 28.2, 24.6, 24.5, 23.4, 22.3, 21.9.

Compound 58

Viscous liquid; ¹H NMR (CDCl₃, 400 MHz) δ 5.15 (1H, d), 4.40 (1H, dd), 4.31 (1H, dd), 3.87-3.86 (1H, m), 3.67 (3H, s), 3.55-3.49 (1H, m), 2.20-1.92 (4H, m), 1.69-1.64 (1H, m), 1.55-1.49 (1H, m), 1.39 (9H, s), 1.12-1.04 (1H, m), 0.89 (3H, d), 0.86 (3H, t); ¹³C NMR (CDCl₃, 100 MHz) δ 172.3, 171.1, 155.7, 79.4, 58.8, 56.2, 52.2, 52.1, 47.1, 37.8, 31.1, 29.1, 28.3, 24.6, 24.0, 15.6, 11.2.

Compound 59

Viscous liquid; ¹H NMR (CDCl₃, 400 MHz) δ 7.22-7.13 (5H, m), 5.38 (1H, d), 4.62 (1H, dd), 4.25 (1H, dd), 3.67 (3H, s), 3.52-3.46 (1H, m), 3.03 (1H, dd), 2.91 (1H, dd), 2.63-2.59 (1H, m), 1.92-1.75 (3H, m), 1.49-1.42 (1H, m), 1.39 (9H, s); ¹³C NMR (CDCl₃, 100 MHz) δ 172.3, 170.2, 154.9, 136.4, 129.4, 128.3, 126.5, 79.6, 59.2, 53.6, 52.5, 46.7, 40.3, 28.9, 28.3, 28.2, 24.4.

Compound 60

Viscous liquid; ¹H NMR (CDCl₃, 400 MHz) δ 5.65 (1H, d), 4.56 (1H, dd), 4.46 (1H, dd), 3.79-3.52 (4H, m), 3.71 (3H, s), 3.35 (1H, s), 2.27-2.17 (1H, m), 2.09-1.93 (3H, m), 1.40 (9H, s); ¹³C NMR (CDCl₃, 100 MHz) δ 172.3, 169.7, 155.8, 80.2, 63.9, 59.4, 53.5, 52.8, 52.3, 47.1, 29.0, 28.2, 24.7, 22.4.

Compound 61

White solid; ¹H NMR (CDCl₃, 400 MHz) δ 7.40 (6H, d), 7.28 (6H, t), 7.21 (3H, t), 5.22 (1H, d), 4.44 (1H, dd), 4.38 (1H, dd), 3.67 (3H, s), 3.61-3.56 (1H, m), 3.13-3.07 (1H, m), 2.59 (1H, dd), 2.42-2.34 (1H, m), 2.13-1.81 (4H, m), 1.42 (9H, s); ¹³C NMR (CDCl₃, 100 MHz) δ 172.2, 169.1, 154.9, 144.5, 129.6, 127.9, 126.7, 79.7, 66.8, 59.0, 52.2, 51.3, 46.8, 34.5, 29.0, 28.3, 24.6.

Compound 62

Viscous liquid; ¹H NMR (CDCl₃, 400 MHz) δ 5.47 (1H, d), 4.45 (1H, dd), 4.33 (1H, dd), 4.09 (1H, dd), 3.81-3.50 (3H, m), 3.69 (3H, s), 2.25-2.17 (1H, m), 2.07-1.90 (3H, m), 1.40 (9H, s), 1.13 (3H, d); ¹³C NMR (CDCl₃, 100 MHz) δ 172.8, 170.9, 156.2, 79.9, 67.4, 59.3, 55.2, 54.0, 52.7, 52.2, 47.2, 38.5, 30.6, 29.6, 24.8, 18.4.

Compound 63

Viscous liquid; ¹H NMR (CDCl₃, 400 MHz) δ 5.29 (1H, d), 4.59 (1H, dd), 4.48 (1H, dd), 3.72-3.62 (2H, m), 3.65 (3H, s), 2.54 (2H, t), 2.19-2.14 (1H, m), 2.06 (3H, s), 2.02-1.88 (4H, m), 1.85-1.77 (1H, m), 1.36 (9H, s); ¹³C NMR (CDCl₃, 100 MHz) δ 172.2, 170.7, 155.4, 79.6, 58.7, 53.4, 50.8, 46.9, 38.5, 32.4, 31.1, 29.7, 28.9, 24.8, 22.1, 15.5.

Compound 64

White solid; ¹H NMR (CDCl₃, 400 MHz) δ 5.56 (1H, d), 4.59-4.54 (2H, m), 3.87-3.84 (1H, m), 3.82-3.76 (1H, m), 3.72 (3H, s), 3.70-3.63 (2H, m), 3.35 (1H, s), 2.25-2.17 (1H, m), 2.03-1.92 (3H, m), 1.40 (9H, s); ¹³C NMR (CDCl₃, 100 MHz) δ 172.9, 170.2, 155.6, 80.0, 63.9, 58.9, 53.3, 52.6, 47.2, 31.0, 28.8, 28.3, 24.8, 22.1.

Compound 65

Viscous liquid; ¹H NMR (CDCl₃, 400 MHz) δ 7.28-7.17 (5H, m), 5.28 (1H, d), 4.64 (1H, dd), 4.47 (1H, dd), 3.70 (3H, s), 3.61-3.51 (2H, m), 3.18-3.12 (1H, m), 3.08 (1H, dd), 2.90 (1H, dd), 2.15-2.07 (1H, m), 1.94-1.83 (3H, m), 1.37 (9H, s); ¹³C NMR (CDCl₃, 100 MHz) δ 172.5, 170.8, 155.3, 136.5, 129.9, 128.5, 126.9, 79.8, 60.6, 59.1, 53.4, 52.4, 47.0, 39.4, 29.2, 28.5, 25.0.

Compound 66

Viscous liquid; ¹H NMR (CDCl₃, 400 MHz) δ 4.56 (1H, dd), 4.47 (1H, dd), 3.67 (3H, s), 3.61-3.32 (4H, m), 2.20-1.72 (8H, m), 1.41 (9H, s); ¹³C NMR (CDCl₃, 100 MHz) δ 172.5, 170.8, 156.0, 79.8, 67.4, 58.8, 55.8, 52.4, 47.2, 38.5, 28.8, 28.2, 24.8, 18.6.

Compound 67

Viscous liquid; ¹H NMR (CDCl₃, 400 MHz) δ 5.54 (1H, d), 4.48 (1H, dd), 4.35 (1H, dd), 4.11-4.04 (1H, m), 3.69 (3H, s), 3.74-3.59 (2H, m), 3.57-3.49 (1H, m), 2.21-2.13 (1H, m), 2.02-1.88 (3H, m), 1.37 (9H, s), 1.17 (3H, d); ¹³C NMR (CDCl₃, 100 MHz) δ 172.5, 170.8, 156.0, 79.8, 67.4, 58.8, 55.8, 52.4, 47.2, 38.5, 28.8, 28.2, 24.8, 18.6.

Compound 68

Viscous liquid; ¹H NMR (CDCl₃, 400 MHz) δ 5.30 (1H, d), 4.54 (1H, dd), 4.45 (1H, dd), 3.72-3.60 (2H, m), 3.63 (3H, s), 2.51 (2H, t), 2.18-2.12 (1H, m), 2.03 (3H, s), 1.99-1.85 (4H, m), 1.82-1.73 (1H, m), 1.33 (9H, s); ¹³C NMR (CDCl₃, 100 MHz) δ 172.2, 170.7, 155.4, 79.6, 58.6, 52.1, 50.8, 46.9, 38.5, 32.4, 29.7, 28.9, 28.2, 24.8, 24.8, 15.5.

Compound 69

Viscous liquid; ¹H NMR (CDCl₃, 400 MHz) δ 5.13 (1H, d), 4.51 (1H, dd), 4.28 (1H, dd), 3.82-3.76 (1H, m), 3.69 (3H, s), 3.66-3.60 (1H, m), 2.22-2.16 (1H, m), 2.03-1.91 (3H, m), 1.75-1.70 (1H, m), 1.61-1.52 (1H, m), 1.39 (9H, s), 1.14-1.06 (1H, m), 0.99 (3H, d), 0.89 (3H, t); ¹³C NMR (CDCl₃, 100 MHz) δ 172.4, 171.4, 155.7, 79.4, 58.8, 56.2, 52.1, 47.1, 37.9, 29.0, 28.3, 24.9, 24.1, 15.2, 11.2.

Compound 70

Viscous liquid; ¹H NMR (CDCl₃, 400 MHz) δ 5.33 (1H, d), 4.51 (1H, dd), 4.45 (1H, t), 3.69 (3H, s), 3.67-3.63 (1H, m), 3.60-3.54 (1H, m), 2.21-2.14 (1H, m), 2.07-1.88 (3H, m), 1.39 (9H, s), 1.33 (3H, d); ¹³C NMR (CDCl₃, 100 MHz) δ 172.4, 171.7, 155.2, 79.5, 58.6, 52.2, 47.7, 46.7, 28.9, 28.3, 24.9, 18.2.

Compound 71

Viscous liquid; ¹H NMR (CDCl₃, 400 MHz) δ 7.60 (1H, s), 7.06 (2H, d), 6.71 (2H, d), 5.31 (1H, d), 4.61 (1H, dd), 4.49 (1H, dd), 3.69 (3H, s), 3.62-3.58 (1H, m), 3.29-3.24 (1H, m), 3.00 (1H, dd), 2.82 (1H, dd), 2.16-2.10 (1H, m), 1.94-1.87 (3H, m), 1.36 (9H, s); ¹³C NMR (CDCl₃, 100 MHz) δ 172.3, 171.0, 155.8, 130.7, 127.0, 115.4, 79.9, 59.1, 53.3, 52.2, 46.9, 38.6, 37.9, 28.9, 28.3, 24.8, 22.2.

Compound 72

Viscous liquid; ¹H NMR (CDCl₃, 400 MHz) δ 6.57 (1H, s), 5.95 (1H, s), 5.62 (1H, d), 4.49-4.42 (2H, m), 3.69 (1H, m), 3.68 (3H, s), 3.03 (1H, s), 2.31-2.26 (2H, m), 2.22-2.14 (1H, m), 2.11-1.78 (5H, m), 1.37 (9H, s); ¹³C NMR (CDCl₃, 100 MHz) δ 175.7, 172.8, 170.8, 155.9, 79.9, 58.8, 54.1, 52.4, 51.0, 47.0, 31.1,2 8.9, 28.8, 28.2, 24.8.

Compound 73

White solid; ¹H NMR (CDCl₃, 400 MHz) δ 8.64 (1H, s), 7.67 (1H, d), 7.33 (1H, d), 7.15 (1H, d), 7.11 (2H, t), 5.35 (1H, d), 4.78 (1H, dd), 4.50 (1H, dd), 3.67 (3H, s), 3.55-3.49 (1H, m), 3.27-3.09 (3H, m), 2.14-2.06 (1H, m), 1.91-1.77 (3H, m), 1.38 (9H, s); ¹³C NMR (CDCl₃, 100 MHz) δ 172.6, 171.2, 155.4, 136.1, 127.8, 123.8, 121.7, 119.3, 118.5, 111.2, 109.7, 79.6, 58.8, 58.7, 52.4, 52.1, 46.8, 38.6, 29.0, 28.5, 28.3, 24.8.

Compound 74

White solid; ¹H NMR (CDCl₃, 400 MHz) δ 7.43 (6H, d), 7.28 (6H, t), 7.21 (3H, t), 5.07 (1H, d), 4.43 (1H, dd), 4.32 (1H, dd), 3.61 (3H, s), 3.48-3.42 (1H, m), 3.12-3.06 (1H, m), 2.51 (1H, d), 2.15-2.05 (1H, m), 1.95-1.75 (3H, m), 1.56-1.48 (1H, m), 1.40 (9H, s); ¹³C NMR (CDCl₃, 100 MHz) δ 172.0, 169.5, 155.2, 144.5, 129.7, 127.9, 126.7, 79.8, 66.9, 59.1, 52.1, 51.6, 46.6, 34.1, 28.9, 28.3, 24.7.

Compound 75

Viscous liquid; ¹H NMR (CDCl₃, 400 MHz) δ 5.13 (1H, d), 4.49 (1H, dd), 4.44 (1H, t), 3.76-3.70 (1H, m), 3.66 (3H, s), 3.58-3.53 (1H, m), 2.21-2.08 (1H, m), 2.05-1.89 (3H, m), 1.75-1.69 (1H, m), 1.46 (2H, t), 1.37 (9H, s), 0.95 (3H, d), 0.91 (3H, d); ¹³C NMR (CDCl₃, 100 MHz) δ 172.4, 171.8, 155.7, 79.4, 58.6, 52.1, 50.3, 46.7, 41.8, 28.9, 28.3, 24.8, 24.5, 23.3, 21.7.

Compound 76

Viscous liquid; ¹H NMR (CDCl₃, 400 MHz) δ 5.19 (1H, d), 4.48 (1H, dd), 4.24 (1H, dd), 3.76-3.70 (1H, m), 3.66 (3H, s), 3.63-3.57 (1H, m), 2.21-2.13 (1H, m), 2.02-1.88 (4H, m), 1.37 (9H, s), 0.98 (3H, d), 0.89 (3H, d); ¹³C NMR (CDCl₃, 100 MHz) δ 172.4, 171.1, 155.8, 79.3, 58.7, 56.8, 52.0, 47.0, 31.2, 28.9, 28.3, 24.9, 19.2, 17.3.

Compound 77

White solid; ¹H NMR (400 MHz, CD₃OD) δ 7.20-7.42 (15H, m), 4.35 (1H, dd, J=8.4, 4.0 Hz), 4.10 (1H, dd, J=9.2, 5.2 Hz), 3.60 (3H, s), 3.31 (1H, m), 3.00 (1H, m), 2.48 (1H, m), 2.56 (1H, m), 2.15 (1H, m), 1.90 (1H, m), 1.88 (2H, m), 1.44 (9H, br s).

Compound 79

Viscous liquid; ¹H-NMR (CD₃OD, 400 MHz) δ 4.45 (1H, dd, J=5.6 Hz and 3.2 Hz), 4.30 (1H, m), 4.13 (m, 1H), 3.73 (3H, s), 1.70 (1H, m), 1.53-1.58 (2H, m), 1.48 (9H, brs), 1.17 (3H, d, J=6.4 Hz), 0.96 (3H, d, J=6.4 Hz), 0.94 (3H, d, J=6.4 Hz); ¹³C NMR (CD₃OD, 100 MHz) δ 174.7, 170.8, 156.4, 79.2, 67.1, 57.6, 53.3, 51.4, 40.5, 27.3, 24.5, 22.0, 20.5, 18.8.

Compound 80

White solid; ¹H NMR (CD₃OD, 400 MHz) δ 4.38 (1H, q, J=6.8 Hz), 4.30 (1H, m), 4.14 (1H, q, J=7.6 Hz), 3.92 (1H, d, J=6.8 Hz), 1.75 (1H, m), 1.53 (1H, m), 1.43 (9H, brs), 1.38 (3H, d, J=6.8 Hz), 1.25 (3H, t, J=7.2 Hz), 1.15 (1H, m), 0.95 (3H, d, J=6.8 Hz), 0.90 (3H, t, J=7.2 Hz); ¹³C NMR (CD₃OD, 100 MHz) δ 172.8, 172.5, 156.4, 79.1, 60.8, 58.8, 48.2, 37.1, 27.3, 24.3, 16.0, 14.4, 13.0, 10.0.

Compound 81

White crystalline solid; ¹H NMR (CD₃OD, 400 MHz) δ 4.17 (1H, t, J=4.4 Hz), 3.95 (2H, dd, J=17.2, 4.4 Hz), 3.75 (2H, d, J=5.2 Hz), 3.71 (3H, s), 1.45 (9H, brs); ¹³C NMR (CD₃OD, 100 MHz) δ 172.3, 170.3, 156.3, 79.4, 61.9, 56.5, 51.3, 40.5, 27.3.

Compound 82

Viscous liquid; ¹H NMR (CD₃OD, 400 MHz) δ 4.41 (1H, q, J=6.8 Hz), 4.16 (2H, q, J=7.6 Hz), 3.77 (2H, dd, J=17.6, 4.8 Hz), 1.44 (9H, brs), 1.37 (3H, d, J=7.2 Hz), 1.25 (3H, t, J=6.8 Hz); ¹³C NMR (CD₃OD, 100 MHz) δ 172.7, 170.8, 156.9, 79.2, 60.9, 48.2, 42.9, 27.3, 16.2, 13.0.

Compound 83

White crystal; ¹H-NMR (CD₃OD, 400 MHz) δ 7.18-7.28 (5H, m), 4.64 (1H, t, J=6.4 Hz), 4.12 (2H, q, J=6.8 Hz), 4.04 (1H, m), 3.15 (1H, dd, J=14.4, 8.0 Hz), 3.0 (1H, dd, J=13.6, 7.6 Hz), 1.62 (1H, m), 1.42 (9H, brs), 1.38-1.36 (2H, m), 1.88 (3H, t, J=7.6 Hz), 0.92 (3H, t, J=6.4 Hz), 0.90 (3H, t, J=6.4 Hz); ¹³C NMR (CD₃OD, 100 MHz) δ 174.0, 171.3.8, 156.2, 136.5, 128.9, 128.0, 126.4, 79.1, 60.9, 53.6, 53.0, 40.8, 37.0, 27.3, 24.4, 21.9, 20.6, 13.0.

Compound 84

White solid; ¹H NMR (CD₃OD, 400 MHz) δ 7.61 (1H, d, J=7.6 Hz), 7.35 (1H, d, J=7.6 Hz), 7.33 (1H, d), 7.16 (1H, s), 7.11 (2H, t, J=7.2 Hz), 4.50 (1H, t, J=7.6 Hz), 4.41 (1H, dd, J=8.0, 3.2 Hz), 3.68 (3H, s), 3.15-3.20 (1H, m), 2.99-3.10 (3H, m), 2.19-2.16 (1H, m), 1.90-1.83 (3H, m), 1.37 (9H, s); ¹³C NMR (CD₃OD, 100 MHz) δ 172.6, 172.1, 156.2, 136.5, 127.4, 123.7, 121.2, 120.9, 118.5, 110.9, 108.9, 79.1, 59.1, 58.8, 52.8, 51.7, 45.9, 29.6, 28.5, 27.2, 24.3.

Compound 85

Viscous liquid; ¹H NMR (CD₃OD, 400 MHz) δ 4.46 (1H, dd, J=8.4, 5.2 Hz), 4.20 (1H, d, J=8.4 Hz), 3.90 (1H, m), 3.67 (1H, m), 3.69 (3H, s), 3.66-3.60 (1H, m), 2.27-2.22 (1H, m), 2.06-1.92 (3H, m), 1.78-1.73 (1H, m), 1.63-1.59 (1H, m), 1.42 (9H, brs), 1.12-1.1.07 (1H, m), 0.99 (3H, d, J=7.2 Hz), 0.89 (3H, t, J=6.8 Hz); ¹³C NMR (CDCl₃, 100 MHz) δ 172.5, 172.1, 156.5, 79.0, 59.0, 56.3, 51.1, 47.1, 36.7, 28.7, 27.3, 24.5, 24.2, 14.0, 9.8.

Compound 86

Viscous liquid; ¹H NMR (CD₃OD, 400 MHz) δ 4.46 (1H, dd, J=4.8, 8.8 Hz), 4.37 (1H, q, J=6.8 Hz), 3.77 (1H, m), 3.69 (3H, s), 3.65-3.62 (1H, m), 3.60-3.54 (1H, m), 2.30-2.23 (1H, m), 2.07-1.93 (3H, m), 1.42 (9H, s), 1.28 (3H, d, J=7.6 Hz); ¹³C NMR (CDCl₃, 100 MHz) δ 172.7, 172.6, 156.2, 79.0, 58.9, 51.3, 47.7, 46.6, 28.5, 27.3, 24.5, 15.7.

Compound 87

Viscous liquid; ¹H NMR (CD₃OD, 400 MHz) δ 4.36 (1H, d, J=6.0 Hz), 3.73 (2H, brs), 3.71 (3H, s), 2.18-2.10 (1H, m), 1.44 (9H, s), 0.94 (3H, d, J=6.8 Hz), 0.93 (3H, d, J=6.4 Hz); ¹³C NMR (CD₃OD, 100 MHz) δ 173.5, 172.6, 159.2, 80.8, 59.0, 52.6, 44.5, 32.1, 28.8, 19.5, 18.4.

Compound 88

Viscous liquid; ¹H NMR (CD₃OD, 400 MHz) δ 4.36 (1H, dd, J=5.6, 2.8 Hz), 3.75 (2H, brs), 3.72 (3H, s), 2.20-2.12 (1H, m), 0.96 (3H, d, J=4.4 Hz), 0.94 (3H, d, J=5.2 Hz); ¹³C NMR (CD₃OD, 100 MHz) δ 171.8, 166.1, 57.8, 51.1, 39.9, 30.4, 17.9 16.8.

Compound 90

White solid, mp 86-88 □; [α]_{D} −13.6 (c 1.0, CH₃OH); ¹H NMR (CD₃OD) δ 7.27 (2H, m), 7.20 (1H, m), 7.18 (2H, m), 4.66 (1H, dd, J=8.0, 5.6 Hz), 4.45 (1H, dd, J=7.6, 6.0 Hz), 3.67 (3H, s), 3.66 (3H, s), 3.13 (1H, dd, J=14.0, 5.6 Hz), 3.02 (1H, dd, J=14.0, 8.0 Hz), 2.73 (1H, dd, J=16.4, 6.0 Hz), 2.59 (1H, dd, J=16.4, 7.6 Hz), 1.42 (9H, s); ¹³C NMR (CD₃OD) δ 171.8, 171.6, 171.1, 156.1, 136.4, 128.9, 128.1, 126.5, 79.5, 53.7, 51.3, 51.0, 48.2, 36.9, 35.8, 27.2.

Compound 91

Viscous light yellow liquid; [α]_{D} −3.73 (c 0.54, CH₃OH); ¹H NMR (CD₃OD) δ 8.24 (1H, s), 7.29 (2H, m), 7.23 (1H, m), 7.20 (2H, m), 4.71 (1H, dd, J=8.4, 5.6 Hz), 4.13 (1H, dd, J=8.8, 4.0 Hz), 3.74 (3H, s), 3.70 (3H, s), 3.20 (1H, dd, J=14.0, 5.6 Hz), 3.02 (1H, dd, J=14.0, 8.8 Hz), 2.96 (1H, dd, J=14.0, 4.0 Hz), 2.82 (1H, dd, J=17.6, 8.8 Hz) ppm; ¹³C NMR (CD₃OD) δ 173.1, 171.8, 169.9, 166.8, 138.0, 130.3, 129.8, 128.2, 55.7, 53.1, 53.0, 51.0, 38.2, 36.6.

Compound 92

White solid; mp 88-91 □; [α]_{D} −11.8 (c 1.0, CH₃OH); ¹H NMR (CD₃OD) δ 7.27 (2H, m), 7.20 (1H, m), 7.18 (2H, m), 5.92 (1H, ddt, J=17.2, 10.8, 6.0 Hz), 5.30 (1H, dq, J=17.2, 1.2 Hz), 5.20 (1H, dd, J=10.8, 1.2 Hz), 4.66 (1H, dd, J=7.6, 5.2 Hz), 4.57 (2H, brd, J=6.0 Hz), 4.47 (1H, dd, J=8.0, 5.2 Hz), 3.67 (3H, s), 3.13 (1H, dd, J=13.2, 5.2 HZ), 3.03 (1H, dd, J=13.2, 7.6 Hz), 2.77 (1H, dd, J=16.4, 5.2 Hz), 2.61 (1H, dd, J=16.4, 8.0 Hz), 1.42 (9H, s) ppm; ¹³C NMR (CD₃OD) δ 171.8, 171.6, 170.3, 156.1, 136.4, 132.1, 128.9, 128.1, 126.5, 117.0, 79.5, 65.1, 53.7, 51.3, 51.0, 36.9, 35.8, 27.3.

Compound 93

Viscous liquid; [α]_{D} −4.063 (c 0.58, CH₃OH); ¹H NMR (CD₃OD) δ 8.24 (1H, s), 7.29 (2H, m), 7.22 (1H, m), 7.20 (2H, m), 5.94 (1H, ddt, J=17.2, 10.4, 6.0 Hz), 5.34 (1H, dq, J=17.2, 1.2 Hz), 5.25 (1H, dd, J=10.4, 1.2 Hz), 4.71 (1H, dd, J=8.4, 5.6 Hz), 4.65 (2H, brd, J=6.0 Hz), 4.14 (1H, dd, J=8.8, 4.0 Hz), 3.70 (3H, s), 3.20 (1H, dd, J=14.0, 5.6 Hz), 3.02 (1H, dd, J=14.0, 8.8 Hz), 2.99 (1H, dd, J=14.0, 4.0 Hz), 2.85 (1H, dd, J=18.0, 9.2 Hz); ¹³C NMR (CD₃OD) δ 173.1, 171.1, 169.8, 166.8, 138.0, 133.3, 130.3, 129.8, 128.2, 119.2, 67.3, 55.7, 53.0, 50.9, 38.2, 36.7.

Compound 94

White solid; mp 55-60 □; [α]_{D} −9.16 (c 1.0, CH₃OH); ¹H NMR (CD₃OD) δ 7.34 (2H, m), 7.27 (2H, m), 7.25 (2H, m), 7.21 (1H, m), 7.20 (1H, m), 7.18 (2H, m), 5.11 (2H, s), 4.66 (1H, dd, J=8.0, 5.6 Hz), 4.45 (1H, dd, 7.6, 6.0 Hz), 3.67 (3H, s), 3.13 (1H, dd, 14.0, 5.6 Hz), 3.02 (1H, dd, 14.0, 8.0 Hz), 2.73 (1H, dd, 16.4, 6.0 Hz), 2.59 (1H, dd, 16.4, 7.6 Hz), 1.42 (9H, s); ¹³C NMR (CD₃OD) δ 171.8, 171.6, 170.4, 156.1, 136.4, 135.9, 128.9, 128.1, 128.1, 127.8, 126.5, 126.5, 79.5, 66.2, 53.7, 51.3, 51.0, 36.9, 35.8, 27.2.

Compound 95

Viscous liquid; ¹H NMR (CD₃OD) δ 8.18 (s), 7.36 (2H, m), 7.27 (4H, m), 7.25 (3H, m), 7.20 (1H, m), 7.20 (2H, m), 5.19 (2H, d, J=2.8 Hz), 4.70 (1H, dd, J=8.8, 5.2 HZ), 4.19 (1H, dd, J=8.8, 4.0 Hz), 3.67 (s), 3.19 (1H, dd, J=14.4, 5.6 Hz), 2.98 (1H, dd, J=14.0, 8.8 Hz), 2.96 (1H, dd, J=18.0, 4.0 Hz), 2.90 (1H, dd, J=18.0, 8.8 Hz); ¹³C NMR (CD₃OD) δ 173.1, 171.6, 169.8, 166.1, 138.0, 137.2, 130.3, 129.8, 129.8, 129.6, 129.6, 128.2, 68.5, 55.7, 53.0, 50.8, 38.1, 36.5

Compound 96

White solid; ¹H NMR (CD₃OD) δ 7.32 (2H, m), 7.32 (1H, m), 7.32 (2H, m), 4.73 (1H, dd, J=7.6, 5.2 Hz), 4.53 (1H, dd, J=8.0, 6.4 Hz), 3.67 (3H, s), 3.65 (3H, s), 3.11 (1H, dd, J=14.0, 5.2 Hz), 3.01 (1H, dd, J=14.0, 7.6 Hz), 2.74 (1H, dd, J=16.4, 6.4 Hz), 2.55 (1H, dd, J=16.4, 8.0 Hz), 1.89 (3H, s); ¹³C-NMR (CD₃OD) δ 171.6, 171.2, 171.2, 168.6, 136.4, 128.8, 128.1, 127.2, 126.4, 53.8, 51.3, 51.0, 50.1, 36.8, 34.9, 22.5

Compound 97

White solid; ¹H NMR(CD₃OD) δ 7.79 (2H, dd, J=6.8, 1.6 Hz), 7.57 (1H, tt, J=7.6, 1.2 Hz), 7.47 (2H, t, J=6.8, 8.0), 7.12 (2H, m), 7.12 (1H, m), 7.12 (2H, m), 4.99 (1H, dd, J=7.6, 5.2 Hz), 4.45 (1H, dd, J=8.0, 6.4 Hz), 3.67 (3H, s), 3.66 (3H, s), 3.14 (1H, dd, J=14.0, 5.2 Hz), 3.00 (1H, dd, J=14.0, 7.6 Hz), 2.94 (1H, dd, J=16.4, 6.4 Hz), 2.78 (1H, dd, J=16.4, 8.0 Hz); ¹³C NMR (CD₃OD) δ 171.6, 171.2, 171.2, 168.6, 136.4, 133.4, 131.6, 128.8, 128.1, 128.1, 127.2, 126.4, 53.8, 51.3, 51.0, 50.1, 36.8, 34.9.

Compound 98

White solid; ¹H NMR (CD₃OD) δ 7.32 (5H, m), 5.92 (1H, ddt, J=17.2, 10.8, 6.0 Hz), 5.30 (1H, dq, J=17.2, 1.2 Hz), 5.20 (1H, dd, J=10.8, 1.2 Hz), 4.80 (1H, dd, J=7.6, 5.2 Hz), 4.66 (1H, dd, J=8.0, 6.4 Hz), 4.57 (2H, brd, J=6.0 Hz), 3.67 (3H, s), 3.14 (1H, dd, J=14.0, 5.2 Hz), 3.00 (1H, dd, J=14.0, 7.6 Hz), 2.83 (1H, dd, J=14.0, 5.2 Hz), 2.64 (1H, dd, J=16.4, 6.4 Hz), 1.89 (3H, s); ¹³C NMR (CD₃OD) δ 171.6, 171.2, 170.3, 168.6, 136.5, 132.1, 129.0, 128.2, 126.6, 117.1, 65.1, 53.8, 51.5, 49.5, 36.9, 35.4, 21.2

Compound 99

Viscous liquid; ¹H NMR (CD₃OD) δ 7.79 (2H, dd, J=6.8, 1.6 Hz), 7.57 (1H, tt, J=7.6, 1.2), 7.47 (2H, dd, J=6.8, 8.0), 7.11 (5H, m), 5.92 (1H, ddt, J=17.2, 10.8, 6.0 Hz), 5.30 (1H, dq, J=17.2, 1.2 Hz), 5.20 (1H, dd, J=10.8, 1.2 Hz), 4.99 (1H, dd, J=7.6, 5.2 Hz), 4.68 (1H, dd, J=8.0, 6.4 Hz), 4.58 (2H, brd, J=6.0 Hz), 3.70 (3H, s), 3.14 (1H, dd, J=14.0, 5.2 Hz), 3.01 (1H, dd, J=14.0, 7.6 Hz), 2.98 (1H, dd, J=14.0, 5.2 Hz), 2.81 (1H, dd, J=16.4, 6.4 Hz); ¹³C-NMR (CD₃OD) δ 171.6, 171.2, 170.4, 168.6, 136.4, 134.0, 133.4, 131.6, 128.8, 128.1, 128.1, 127.2, 126.4, 119.0, 65.1, 53.8, 51.3, 51.0, 36.8, 35.0

Compound 100

White solid; ¹H NMR (CD₃OD) δ 7.32 (5H, m), 7.21 (5H, m), 5.11 (2H, s), 4.78 (1H, dd, J=7.6, 5.2 Hz), 4.63 (1H, dd, J=8.0, 6.4 Hz), 3.67 (3H, s), 3.13 (1H, dd, J=14.0, 5.2 Hz), 3.00 (1H, dd, J=14.0, 7.6 Hz), 2.84 (1H, dd, J=14.0, 5.2 Hz), 2.65 (1H, dd, J=16.4, 6.4 Hz), 1.89 (3H, s); ¹³C NMR (CD₃OD) δ 171.8, 171.6, 171.2, 170.2, 168.6, 136.5, 135.9, 128.9, 128.1, 128.1, 127.9, 127.8, 126.5, 66.1, 53.8, 51.3, 49.6, 36.8, 35.4, 21.0

Compound 101

White solid; ¹H NMR(CD₃OD) δ 7.79 (2H, dd, J=6.8, 1.6 Hz), 7.57 (1H, tt, J=7.6, 1.2 Hz), 7.47 (2H, dd, J=6.8, 8.0 Hz), 7.34 (2H, m), 7.25 (4H, m), 7.20 (1H, m), 7.12 (5H, m), 5.13 (2H, s), 4.99 (1H, dd, J=7.6, 5.2 Hz), 4.45 (dd, J=8.0, 6.4 Hz), 3.69 (s), 3.14 (dd, J=14.0, 5.2 Hz), 3.00 (dd, J=14.0, 5.2 Hz), 2.94 (dd, J=16.4, 6.4 Hz), 2.78 (dd, J=6.4, 8.0 Hz); ¹³C NMR (CD₃OD) δ 171.6, 171.2, 170.5, 168.6, 136.3, 135.9, 133.4, 131.6, 128.8, 128.1, 128.1, 128.1, 127.9, 127.8, 127.2, 126.4, 66.3, 53.8, 51.3, 50.1, 36.8, 35.2

Compound 102

White solid; ¹H NMR (CD₃OD) δ 7.15 (3H, m), 7.04 (2H, m), 4.65 (1H, dd, J=7.6, 5.2 Hz), 3.79 (1H, dd, J=8.0, 6.4 Hz), 3.61 (3H, s), 3.11 (1H, dd, J=14.0, 5.2 Hz), 3.01 (1H, dd, J=14.0, 7.6 Hz), 2.74 (1H, dd, J=16.4, 6.4 Hz), 2.55 (1H, dd, J=16.4, 8.0 Hz), 1.89 (3H, s); ¹³C-NMR (CD₃OD) δ 173.1, 171.6, 171.1, 170.7, 135.8, 129.1, 128.4, 126.9, 53.5, 52.2, 49.1, 37.4, 35.3, 22.4.

Compound 103

White solid; Melting point 180-190 □ (dec); ¹H NMR (CDCl₃, 400 MHz) δ 7.23 (1H, brt), 6.65 (1H, d, J=2.2 Hz), 4.39 (1H, t, J=8.8 Hz), 4.08 (1H, dd, J=18, 6.0 Hz) 3.91 (1H, dd, J=18, 6.0 Hz), 3.72 (3H, s), 2.06 (1H, m), 2.0 (3H, s), 0.96 (3H, d, J=7.6 Hz), 0.94 (3H, d, J=7.6 Hz) ppm; ¹³C NMR (100 MHz, CDCl₃) δ 172.2, 170.6, 170.2, 58.5, 52.5, 41.3, 31.3, 23.2, 19.3, 18.5 ppm.

Compound 104

White solid; ¹H NMR (CDCl₃, 400 MHz) δ 7.17 (1H, brt), 6.55 (1H, d, J=8.4 Hz), 4.56 (1H, m), 4.03 (1H, dd, J=18.4, 6.0 Hz) 3.93 (1H, dd, J=18.0, 5.2 Hz), 3.72 (3H, s), 1.97 (3H, s), 1.66 (2H, m), 1.54 (1H, m), 0.92 (3H, d, J=7.6 Hz), 0.90 (3H, d, J=7.6 Hz) ppm; ¹³C NMR (100 MHz, CDCl₃) δ 172.8, 170.5, 170.1, 52.3, 51.5, 41.1, 41.0, 24.7, 23.0, 22.8, 22.2 ppm.

Compound 105

White solid; ¹H NMR (CDCl₃, 400 MHz) δ 7.57 (1H, brt), 4.60 (1H, dd, J=7.2 Hz), 3.97 (2H, d, J=6.0 Hz), 3.70 (3H, s), 3.55 (1H, m), 3.41 (1H, m), 2.43 □ 2.38 (1H. m), 2.10 (3H, s), 2.01 □ 1.93 (2H, m), 1.90 □ 1.80 (1H, m) ppm; ¹³C NMR (100 MHz, CDCl₃) δ 171.5, 171.1, 170.1, 59.3, 52.2, 48.2, 41.2, 27.3, 24.9, 22.5 ppm.

Compound 106

Viscous liquid; ¹H NMR (CDCl₃, 400 MHz) δ 5.39 (1H, s), 4.50 (1H, dd), 3.94 (1H, dd), 3.69 (3H, s), 3.59-3.55 (1H, m), 3.46-3.40 (1H, m), 2.21-2.14 (1H, m), 2.10-1.94 (4H, m), 1.40 (9H, s); ¹³C NMR (CDCl₃, 100 MHz) δ 172.8, 167.7, 156.2, 80.0, 58.8, 52.7, 46.2, 43.4, 29.4, 28.7, 25.0.

Compound 107

Viscous liquid; ¹H NMR (CD₃OD, 400 MHz) δ 7.52 (1H, d, J=7.6 Hz), 7.33 (1H, d, J=7.6 Hz), 7.10 (1H, d), 7.08 (1H, s), 7.01 (2H, t, J=7.2 Hz), 4.80 (1H, t, J=7.6 Hz), 4.20 (1H, dd, J=8.0, 3.2 Hz), 3.68 (3H, s), 3.39-3.26 (1H, m), 3.22-3.17 (3H, m), 2.38 (H, m), 2.03 (3H, 1H); ¹³C NMR (CD₃OD, 100 MHz) δ 173.6, 169.9, 138.2, 128.7, 124.7, 122.7, 120.0, 119.7, 112.6, 110.6, 61.0, 60.9, 55.4, 53.0, 47.6, 31.2, 28.5, 25.0.

Compound 108

Viscous liquid; ¹H NMR (CD₃OD, 400 MHz) δ 4.47 (1H, q), 3.70 (3H, s), 3.68 (1H, d), 2.21 (1H, m), 1.42 (3H, s), 1.09 (6H, d); ¹³C NMR (CD₃OD, 100 MHz) δ 174.2, 169,5, 59.7, 52.9, 49.7, 31.7, 18.8, 18.0, 17.3.

Compound 109

White solid; ¹H NMR (CD₃OD, 400 MHz) δ 4.90 (1H, q, J=6.8 Hz), 4.84 (1H, m), 4.47 (1H, q, J=7.6 Hz), 3.70 (1H, d, J=6.8 Hz), 1.94 (1H, m), 1.61 (1H, m), 1.42 (3H, d, J=6.8 Hz), 1.22 (1H, m), 1.07 (3H, d, J=6.8 Hz), 0.99 (3H, t, J=7.2 Hz); ¹³C NMR (CD₃OD, 100 MHz) δ 174.7, 169.5, 59.1, 52.9, 49.7, 38.3, 25.4, 17.3, 15.1, 11.8.

### Example 1

### Expression of Defense Gene in Pepper Plant

In order to testify the disease resistance expression in the case of treating the dipeptide derivative of the present invention to the seeding of pepper, PR-1, beta-1,3-glucanase, chitinase, PR4, peroxidase, and PR10 primer, and the seedling of pepper were used. The seedling of pepper was raised in a greenhouse for 4 weeks, Compounds were sprayed thereto in various concentrations, and then, after three or one week, phytophthora (Phytophthora capcisi) and the pathogen of a bacterial rot (Pectobacterium carotovorum) were respectively inoculated.

Since then, after 12, 24, and 48 hours, the respective pepper leaves were collected in an amount of 1 g, and then, stored at an ultralow temperature-room at 75° C. The stored plant leaves were pulverized along with liquid nitrogen, and then, rRNA was extracted by using a RNA extraction kit (Easy-Spin™IIP Total RNA Extraction Kit, Intron Biotechnology, Korea) and Ex Taq polymerase by Kishimoto et al. (2005) (Takara Biomedicals, Otsu, Japan). For amplification, 0.1 μg of cDNA, 10 pMol of each of forward and reverse primers, 250 nM of dNTPs, and 0.5 U of Ex Taq polymerase were added to 20 μl, of a buffer solution. PCR was performed at 94C and 58C in a thermal cycler (PTC-100, USA), and the products by PCR were analyzed under the condition of 1% agarose gel (in 0.5×TAE buffer) (80 V, 60 min). All the RT-PCR tests were repeated twice.

The results thus obtained are illustrated in FIG. 1. According to FIG. 1, it can be confirmed that when Compound 80 or 89 was treated and then P. carotovora or P. capsici was treated, the genes of PR1, PR4, PR10, beta-1,3-glucanase, chitonase, and peroxidase were strongly expressed.

### Example 2

### Measurement of Degrees of PR-1 Gus Activation on Tobacco Leaves

After the tobacco (Xanth-nc) bound with GUS gene induced by PR-1α disease-resistance promoter was grown for 3 weeks, 100 μL of the diluted solution of Compound 74 was injected to second leaves with a syringe. After three days, the surrounding leaves were collected in an internal diameter size of 5 mm with a cork borer, the samples thus obtained were added to a 1.5 mL of an eppendorf tube, and 20 μL of GUS extraction buffer solution was further added, ground, and then centrifuged at 8000 G for 3 minutes to obtain supernatant. The same amount of 2 mM MUG (4-methyl umbellifryl-B-glucuronide) solution was added, reacted at 37° C. for 1 hour, and then 960 μL of stop buffer solution (0.2 M, Na₂CO₃ solution) was added to be 1 mL. Since then, the fluorescence was measured with a TKO 100 fluorometer (Hoefoer Scientific Instruments, USA). At this time, the fluorescence amount was calibrated using a MU (4-methyl umbellyferon) as a standard reagent, and the GUS activity was calculated using MU-mM/sample−10 mg/time. The results thus obtained are listed in the following Table 2 and illustrated in FIG. 2.

TABLE 2
Degree of expression of PR-1a GUS
promoter by treating Compound 74

| | | | PR1a GUS expression |
|---|---|---|---|
| Test substance | | | (nM MU/10 mg FW/1 hr) |
| Control group | | | 539.0 |
| 0.1 mM BTH | | | 324,000 |
| Compound 74 | 0.1 | ppm | 7,800 |
| | 1.0 | ppm | 15,000 |
| | 10.0 | ppm | 420,000 |
| | 100.0 | ppm | 860,000 |

From the above Table 2, it can be confirmed that as a result of coloring GUS expressed in the case of treating Compound 74 to a tobacco, in the case of treating it in the concentration of 100 ppm, the expression amount is the biggest.

### Example 3

### Effect on Suppressing Outbreak of Plant Disease

After treating the compounds of the present invention to various plants, the effects of the compounds on suppressing the outbreak of plant disease were measured in the following method.

A tobacco or cucumber was sown in a plastic pot with a diameter of 10 cm×13 cm, and then, 12 pots were added in a yellow box. After seeding, 100 μL was inoculated to the cotyledons of cucumber or tobacco, in which first leaf was started to come out. The test compounds were dissolved in 20% methanol to make a stock solution, and then, the stock solution was diluted to the concentrations of 1, 10, and 100 ppm.

After 7 days, the pathogen of a plant bacterial rot (Pectobacterium carotovorum SCC1) was cultured in a TSA medium for 27 hours; and then, the cultured pathogen was sprayed in a concentration of 10⁸ cfu/mL to the plant leaves, and cultured at 30° C. for 3 days. The plant anthracnose germ (Colletotrichum orbiculare) was cultured in a GBA (Green bean agar) medium to induce spores for about 2 to 3 weeks. The germ was sprayed in a concentration of 10⁵ cell/mL and then cultured at 26° C. for 1 day. When fourth or fifth leaf came out, 200 μL of each of the test compounds was inoculated to third leaf. After 7 days, the pathogen of the plant bacterial rot was sprayed to the plant leaves in the concentration of 10⁸ cfu/mL, and then cultured at 30° C. for 3 days.

The investigation was performed with naked eyes, and the lesion relative areas were investigated until 0 to 100% according to the shortness degrees of the bacterial rot. The anthracnose was investigated by counting the number of lesions occurred on the leaves.

The test results of the effect on suppressing the outbreak of a bacterial rot to a tobacco plant are illustrated in the photographs of FIG. 3, and listed in the following Tables 3 to 6.

TABLE 3
Effect on suppressing tobacco bacterial
rot (Pectobacterium carotovorum)

| | Lesion outbreak rate (%) | | |
|---|---|---|---|
| Test substance | 1 ppm | 10 ppm | 100 ppm |
| Compound 1 | 73 | 10 | 57 |
| Compound 2 | 60 | 39 | 41 |
| Compound 3 | 13 | 68 | 80 |
| Compound 4 | 47 | 80 | 90 |
| Compound 5 | 73 | 40 | 7.7 |
| Compound 6 | 11 | 2.1 | 3.5 |
| Compound 7 | 40 | 27 | 20 |
| Compound 8 | 43 | 57 | 0 |
| Compound 9 | 90 | 53 | 82 |
| Compound 10 | 80 | 73 | 87 |
| Compound 11 | 30 | 46 | 21 |
| BTH* | 90 | 90 | 90 |
| Non-treated group | 100 | 100 | 100 |
| LSD (p = 0.05) | 50 | 39 | 38 |

BTH: benzo-1,2,3-thiadiazole-7-carbothio acid S-methyl ester

TABLE 4
Effect on suppressing tobacco bacterial
rot (Pectobacterium carotovorum)

| | Lesion outbreak rate (%) | | |
|---|---|---|---|
| Test substance | 1 ppm | 10 ppm | 100 ppm |
| Compound 24 | 13 | 65 | 33 |
| Compound 25 | 50 | 70 | 50 |
| Compound 26 | 63 | 23 | 60 |
| Compound 27 | 33 | 30 | 20 |
| Compound 28 | 20 | 80 | 13 |
| Compound 29 | 63 | 43 | 63 |
| Compound 30 | 23 | 36 | 13 |
| Compound 31 | 63 | 56 | 73 |
| Compound 32 | 78 | 30 | 60 |
| Compound 33 | 80 | 38 | 18 |
| Compound 34 | 35 | 41 | 24 |
| Compound 35 | 18 | 43 | 50 |
| Compound 36 | 45 | 41 | 38 |
| Compound 37 | 5 | 5 | 43 |
| Compound 38 | 13 | 80 | 60 |
| Compound 39 | 35 | 10 | 43 |
| Compound 40 | 8 | 45 | 43 |
| Compound 41 | 30 | 50 | 18 |
| Compound 42 | 43 | 60 | 45 |
| Compound 43 | 35 | 45 | 31 |
| Compound 44 | 46 | 37 | 33 |
| Compound 45 | 83 | 33 | 55 |
| Compound 46 | 56 | 70 | 48 |
| BTH | 88 | 88 | 88 |
| CON | 100 | 100 | 100 |
| LSD (p = 0.05) | 37 | 48 | 48 |

TABLE 5
Effect on suppressing tobacco bacterial
rot (Pectobacterium carotovorum)

Lesion outbreak rate (%)

| Test substance | 10 ppm | 100 ppm |
|---|---|---|
| Compound 47 | 0 | 50 |
| Compound 48 | 25 | 50 |
| Compound 49 | 23 | 0 |
| Compound 50 | 35 | 60 |
| Compound 51 | 0 | 68 |
| Compound 52 | 0 | 25 |
| Compound 53 | 45 | 50 |
| Compound 54 | 36 | 60 |
| Compound 55 | 48 | 36 |
| Compound 56 | 13 | 40 |
| Compound 57 | 25 | 25 |
| Compound 58 | 28 | 13 |
| Compound 59 | 38 | 25 |
| Compound 60 | 18 | 56 |
| Compound 61 | 20 | 16 |
| Compound 62 | 45 | 41 |
| Compound 63 | 56 | 52 |
| BTH | 25 | 25 |
| CON | 100 | 100 |
| LSD (p = 0.05) | 58 | 56 |

TABLE 6
Effect on suppressing tobacco bacterial
rot (Pectobacterium carotovorum)

| | Lesion outbreak rate (%) | | |
|---|---|---|---|
| Test substance | 1 | 10 | 100 ppm |
| Compound 64 | 100 | 23 | 100 |
| Compound 65 | 80 | 57 | 55 |
| Compound 66 | 63 | 58 | 53 |
| Compound 67 | 28 | 23 | 88 |
| Compound 68 | 78 | 53 | 25 |
| Compound 69 | 63 | 56 | 59 |
| Compound 70 | 80 | 72 | 77 |
| Compound 71 | 60 | 65 | 40 |
| Compound 72 | 55 | 72 | 45 |
| Compound 73 | 35 | 60 | 46 |
| Compound 74 | 25 | 34 | 22 |
| Compound 75 | 25 | 80 | 100 |
| Compound 76 | 46 | 41 | 37 |
| Compound 12 | 45 | 33 | 25 |
| Compound 13 | 100 | 100 | 28 |
| Compound 14 | 50 | 78 | 35 |
| Compound 15 | 75 | 63 | 50 |
| Compound 16 | 25 | 25 | 53 |
| Compound 17 | 100 | 25 | 100 |
| Compound 18 | 80 | 75 | 68 |
| Compound 19 | 49 | 42 | 50 |
| Compound 20 | 56 | 63 | 51 |
| Compound 21 | 37 | 46 | 35 |
| Compound 22 | 35 | 24 | 46 |
| Compound 23 | 25 | 52 | 36 |
| BTH | 100 | 100 | 100 |
| CON | 100 | 100 | 100 |
| LSD (p = 0.05) | 37 | 37 | 38 |

### Example 4

### Effect on Suppressing Outbreak of Disease on Cucumber Leaves by Seed Treatment

Compound 61 or Compound 74 were deposited on the seeds of cucumbers in the concentration of 100 ppm for 2 hours, and after 3 weeks, the spores of anthracnose germ (Colletotrichum orbiculare) were sprayed in the concentration of 10⁵ spore/mL thereto, and then, after 7 days, the disease outbreak was investigated. The results thus obtained are illustrated in the photographs of FIG. 4.

According to FIG. 4, it can be confirmed that the anthracnose was hardly found on the leaves of cucumber treated with Compound 61 or Compound 74 as compared with the non-treated group.

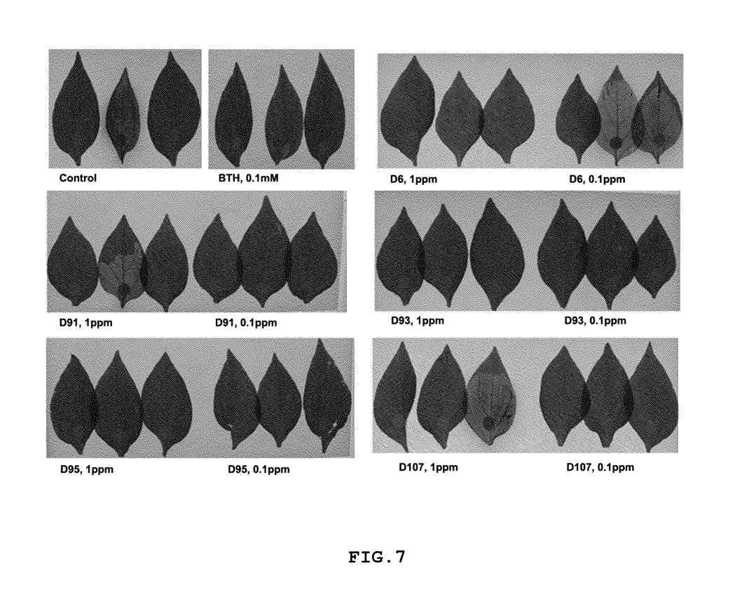

The peppers were raised in the same method as Example 3, and then, the test compounds were dissolved in 20% methanol to make a stock solution, and a stock was diluted to the concentrations of 0.1, 1.0, and 10.0 ppm. The test solutions were sprayed on the leaves or drench-treated on the leaves. After 7 days, the pathogen of plant bacterial rot (Pectobacterium carotovorum SCC1) was cultured in a TSA medium for 27 hours, and then, wetted on an 8 mm diameter paper disc in the concentration of 10⁸ cfu/mL. The paper disc was located in the center of the leaves that were cut roundly, and then, after about 7 days, the lesion area was measured. In the case of phytophthora, the potato agar medium cultured with the phytophthora (Phytophthora capsici) was cut in the internal diameter size of 5 mm with a cork borer, and then inoculated to the leaves. After 7 days, the lesion area was observed. In the case of a bacterial rot, when Compounds 6, 89, 93, and 95 were sprayed on the pepper leaves, the bacterial rot was not found in the concentration of 1 or 10 ppm as compared with the non-treated group. In addition, for the pepper phytophthora test, when Compounds 89, 94, 95, and 107 were drench-treated in the concentration of 1.0 or 10.0 ppm and Compound 107 (1 or 0.1 ppm) was sprayed on the leaves, there were no the lesion formations. Especially, it could be confirmed that Compound 89 exhibited the excellent effect on suppression the outbreak of the pepper phytophthora blight as compared with the control drug, BTH (see FIG. 7). The above-described test results indicate that these compounds induce the disease resistance in a plant and suppress the generation of the pathogen of a bacterial rot or phytophthora, thus suggesting that these substances can be used as an agricultural germicide. The Suppression of lesion formation on the leaves when these compounds were drench-treated to the roots can be explained as it was resulted from that systemic disease resistance was induced in the whole plant. These test results are illustrated in the photographs of FIGS. 5 to 9.

TABLE 7
Effect on suppressing pepper phytophthora
blight (Phytopthora capsici)

| | Lesion outbreak rate (area %) | | |
|---|---|---|---|
| Test substance | 0.1 | 1.0 | 10.0 ppm |
| Compound 77 | 46 | 36 | — |
| Compound 78 | 58 | 47 | — |
| Compound 79 | 68 | 46 | — |
| Compound 80 | 14 | 8.9 | — |
| Compound 81 | 68 | 58 | — |
| Compound 82 | 74 | 60 | — |
| Compound 83 | 80 | 57 | — |
| Compound 84 | 35 | 33 | — |
| Compound 85 | 24 | 15 | — |
| Compound 86 | 58 | 83 | — |
| Compound 87 | 62 | 36 | — |
| Compound 88 | 35 | 33 | — |
| Compound 89 | 9.3 | 2.3 | — |
| Compound 90 | 24 | 7.2 | — |
| Compound 91 | 14 | 24 | — |
| Compound 92 | 25 | 38 | — |
| Compound 93 | 35 | 23 | — |
| Compound 94 | 12 | 6.8 | — |
| Compound 95 | 9.2 | 3.8 | — |
| Compound 96 | 32 | 23 | — |
| Compound 97 | 22 | 25 | — |
| Compound 98 | 16 | 12 | — |
| Compound 99 | 36 | 18 | — |
| Compound 100 | 19 | 21 | — |
| Compound 101 | 11 | 27 | — |
| Compound 102 | 20 | 15 | — |
| Compound 103 | 15 | 28 | — |
| Compound 104 | 22 | 18 | — |
| Compound 105 | 18 | 21 | — |
| Compound 106 | 25 | 26 | — |
| Compound 107 | 9.3 | 3.5 | — |
| Compound 108 | 14 | 18 | — |
| Compound 109 | 25 | 9.4 | — |
| BTH (0.1 mM) | 17 | | |
| Non-treated control group | 100 | 100 | 100 |
| LSD (p = 0.05) | 88 | 88 | 88 |

### Example 6

### Effect on Suppressing Phytophthora Blight in Pepper Roots

The peppers were raised in the same method as Example 3, Compound 85 was sprayed on the leaves, and then after 1 week, the Phytophthora capsici swarm suspension was drench-inoculated. While observing for 7 days, the lesion formation on the leaves and roots were investigated. As compared with the control group, for the peppers treated with Compound 85, the phytophthora blight on the leaves and roots was significantly suppressed. The test results of Compound 85 on suppressing the outbreak of phytophthora blight are illustrated in FIG. 10.

### Example 7

### Effect on Promoting Plant Growth

The active ingredients (Compounds 5 to 8, 14, and 15) were drenched to the leaves of a plant (pepper, potato, tomato, tobacco, cucumber, and Chinese cabbage), and then, after 7 days, the plant lengths were measured. Since then, the plant disease pathogen was inoculated, and then, after 3 to 5 days, the sizes of the plant leaves were measured and compared with the control group to obtain the relative growth degrees. The effects on promoting the growth of the cucumber leaves are listed in the following Table 8.

TABLE 8

| | Width of cucumber leaf* (mm) | | |
|---|---|---|---|
| Test substance | 100 ppm | 10 ppm | 1.0 ppm |
| Compound 5 | 121.1 | 110.7 | 100.5 |
| Compound 6 | 122.8 | 124.4 | 113.5 |
| Compound 7 | 121.4 | 120.6 | 110.2 |
| Compound 8 | 119.8 | 123.4 | 121.6 |
| Compound 14 | 112.8 | 103.3 | 104.2 |
| Compound 15 | 112.4 | 113.3 | 98.9 |
| Non-treated | 100.0 | 100.0 | 100.0 |

*The width of leaf in the non-treated group is 92.1 mm, and the suggested value is the relative value to the non-treated group.

According to the results listed in the above-described Table 8, it can be confirmed that when the dipeptide derivative of the present invention is treated, the growth of the cucumber is promoted. Therefore, it can be confirmed that the compounds of the present invention induce the plant disease-resistance and also the plant growth-promoting effect. It can be also considered that the growth-promoting ability can attenuate plant-protecting ability from the disease.

### Example 8

### Effect on Overcoming Cold Weather-Damage on Pepper Plant

In order to confirm the immunity-activating effect of the compounds of the present invention, the plants were exposed at the temperature so that they could be artificially damaged from cold weather, and the growth and development were observed. After growing the plants for 6 weeks, the compounds were drenched or sprayed on the leaves and the plants were raised for 1 week. After the plants were exposed under a 2° C. growth room for 1 day, they were raised at room temperature for 3 days. The degrees of the growth and development of the plants were observed and the degrees of the damage from cold weather in the plant were determined. When Compound 95 was drench-treated to the plants, there were no cold weather-damages. When sprayed on the leaves, as with Compounds 90, 91, 93, and 95, there were no damages observed from cold weather in the plant. From the test results, it can be confirmed that when the compounds are applied, the plant exhibits the abiotic resistance and also induces the immunity-activating effect capable of maintaining the growth and development even under the physical harmful environment, like cold weather. The test results are illustrated in the photographs of FIGS. 11 and 12.

### Formulation Examples

Formulation Examples are the representative examples for formulating the agricultural plant-protecting agent including the compound represented by the above-described chemical formula 1 as an active gradient to be suitable for being applied. The compositions of the respective used components used for the formulation are as follows.

### Formulation Example 1

### Water Dispersible Powder

10 g of the compound of chemical formula 1, 10 g of NK250L (surfactant), 10 g of white carbon, and 70 g of pyrophylite (bulking agent) were ground and mixed to prepare water dispersible powder.

### Formulation Example 2

### Emulsion

10 g of the compound of chemical formula 1, 10 g of DDY2000 (surfactant), and 80 g of xylene were mixed to prepare an emulsion.

### Formulation Example 3

### Liquid Water Dispersible Powder

10 g of the compound of chemical formula 1, 10 g of HY1910 (surfactant), 5 g of propyleneglycol, 0.2 g of xanthan gum, 0.15 g of KM-73 (antifoamer), 0.2 g of Biocide-LS (preservative), 0.1 g of KNP (viscosity agent), and 74.35 g of water (bulking agent) were ground in a ball mill and mixed to prepare the liquid water dispersible powder.

### Formulation Example 4

### Water Floating Granule

5 g of the compound of chemical formula 1, 7.5 g of paraffin oil, 2 g of sodiumalkylsulfosuccinate (surfactant), 3 g of white carbon, 1.2 g of xanthan gum, 0.8 g of sodium polyacrylic acid, and 80.5 g of potassium chloride were mixed, granulated into a horizontal extruder, and then, dried to prepare the water floating granule.

### Formulation Example 5

### Granule

5 g of the compound of chemical formula 1, 2.5 g of HY1910 (surfactant), 0.2 g of NK250L (surfactant), 0.5 g of soda ash, 2.0 g of dextrin, 25 g of bentonite, and 64.8 g of talc were mixed with water to make the dough, granulated into a horizontal extruder, and then, dried to prepare the granule.

### Formulation Example 6

### Mixed Formulation

Within the limit range of 20 wt % of the compound of chemical formula 1 used in Formulation Examples 1 to 5 as an active ingredient, the single or mixture selected from the general germicides, insecticides, and herbicides was used to prepare the mixed formulation.

### INDUSTRIAL APPLICABILITY

As described above, in the case of treating the agricultural plant-protecting agent of the present invention to various dicotyledoneaes, such as, peppers, cucumbers, potatoes, and tomatoes, the growth of the plant is promoted, and also, the effect on preventing plant diseases such as bacterial rot, damping-off, phytophthora blight, a wilt disease, a leaf spot disease or a mosaic disease, which are caused by a bacteria, a virus, or a mold, is exhibited. Even though it is not directly applied to the lesion area of the plant, the same effect as described above is exhibited on other part of the plant. In addition, since the agricultural plant-protecting agent of the present invention exhibits the effect on preventing the cold weather-damage so as for the plant to be not damaged from a low temperature, it exhibits the plant immunity-activating effect so as to make the plant stays to grow healthy.

Therefore, the agricultural plant-protecting agent of the present invention can be applied for a plant (in detail, dicotyledoneae) for preventing or suppressing the plant disease, promoting the plant growth, or activating the plant immunity.

1. An agricultural plant-protecting agent comprising of a compound selected from dipeptide derivatives represented by the following chemical formula 1 or an agro-pharmaceutically acceptable salt thereof as an active ingredient: 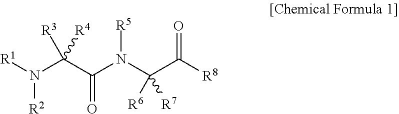
In the above Chemical Formula 1,
R¹, R², and R⁵ are the same or different from each other, and represent a hydrogen atom, a linear or branched C₁-C₁₈ alkyl carbonyl group, a linear or branched C₁-C₁₈ alkoxy carbonyl group, 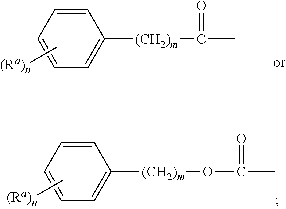
R³, R⁴, R⁶, and R⁷ are the same or different from each other, and represent a hydrogen atom, or a linear or branched C₁-C₁₈ alkyl group substituted or unsubstituted with the group selected from hydroxy, mercapto, amino, guanidino, N,N-bis(benzyloxycarbonyl)guanidino, carbamoyl, carboxylic acid, linear or branched C₁-C₁₈ alkoxy carbonyl, linear or branched C₁-C₁₈ alkenyl oxycarbonyl, 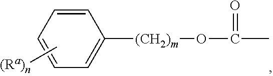
linear or branched C₁-C₁₈ alkyl thio, trityl thio, acetyl amino, phenyl, hydroxyphenyl, imidazole, and indolyl;
or any one of the R³ and R⁴ may bind to R² to form a nitrogen-containing 5-membered to 7-membered ring or any one of the R⁶ and R⁷ may bind to R⁵ to form a nitrogen-containing 5-membered to 7-membered ring.
R⁸ represents hydroxy; a linear or branched C₁-C₁₈ alkoxy group; a linear or branched C₁-C₁₈ alkyl amino group; 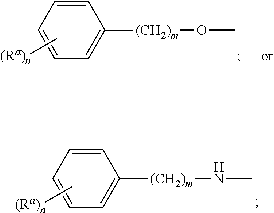
R^{a} represents a linear or branched C₁-C₁₈ alkyl group;
n represents an integer of 0 to 5, the number of a substituent, R^{a}; and
m represents an integer of 0 to 6.]

2. The agricultural plant-protecting agent, according to claim 1, wherein the dipeptide derivatives are represented by the above-described chemical formula 1 is a racemic mixture or isomeric compound.

3. The agricultural plant-protecting agent according to claim 1, wherein the R¹, R², and R⁵ are the same or different from each other, and represent a hydrogen atom, an acetyl group, a hexanoyl group, a hexadecanoyl group, an octadecanoyl group, a benzoyl group, a 4-hexylbenzoyl group, a 2-phenylacetyl group, a 3-phenylpropanonyl group, a methoxycarbonyl group, an ethoxycarbonyl group, a t-butoxycarbonyl group, a hexadecanoxy carbonyl group, an octadecanoxycarbonyl group, a phenoxycarbonyl group, and a 4-hexylbenzyloxycarbonyl group; the R³, R⁴, R⁶, and R⁷ are the same or different from each other, and represent a hydrogen atom, a methyl group, an ethyl group, a n-propyl group, an isopropyl group, a 1-methylpropyl group, a 2-methylpropyl group, a hydroxymethyl group, a 1-hydroxyethyl group, a 2-hydroxyethyl group, an imidazole-4-yl-methyl group, a 2-methylthioethyl group, a benzyl group, a 4-hydroxybenzyl group, a phenethyl group, a mercaptomethyl group, a methylthiomethyl group, a methylthioethyl group, a tritylthiomethyl group, a tritylthioethyl group, a methoxycarbonylmethyl group, an ethoxycarbonylmethyl group, a propanoxycarbonylmethyl group, a tert-butoxycarbonylmethyl group, a pentaneoxycarbonylmethyl group, a hexaneoxycarbonylmethyl group, an allyloxycarbonylmethyl group, a 2-allyloxycarbonylethyl group, a benzyloxycarbonylmethyl group, a benzyloxycarbonylethyl group, a penethyloxycarbonylmethyl group, a 2-penethyloxycarbonylethyl group, a 3-phenylpropyloxycarbonylmethyl group, a 2-(3-phenylpropyloxycarbonyl)ethyl group, a 1-methoxycarbonylethyl group, a 2-methoxycarbonylethyl group, a 2-ethoxycarbonylethyl group, a 2-propaneoxycarbonylethyl group, a 2-butoxycarbonylethyl group, a 2-pentaneoxycarbonylethyl group, a 2-hexaneoxycarbonylethyl group, a 2-aminoethyl group, a carbamoylmethyl group, an acetylaminomethyl group, an acetylaminoethyl group, a carboxymethyl group, a carboxyethyl group, an imidazole-4-ylmethyl group, an imidazole-4-ylethyl group, a 3-guanidinopropyl, a N,N-bis(benzyloxycarbonyl)guanidinopropyl, an indole-3-ylmethyl group, or an indole-3-ylethyl group; or any one of the R³ and R⁴ may bind to R² to form a nitrogen-containing 5-membered ring or any one of the R⁶ and R⁷ may bind to R⁵ to form a nitrogen-containing 5-membered ring; and the R⁸ represents the compound representing a hydrogen atom, a methoxy group, an ethyoxy group, a propaneoxy group, a t-butoxy group, a hexaneoxy group, a hexadecaneoxy group, an octadecaneoxy group, a benzyloxy group, a penethyloxy group, a 3-phenylpropaneoxy group, a methylamino group, an ethylamino group, a propylamino group, a butylamino group, a hexylamino group, a phenylamino group, a benzylamino group, a hexadecylamino group, and an octadecylamino group.

4. The agricultural plant-protecting agent according to claim 1, wherein the dipeptide derivatives represented by the above-described chemical formula 1 is a compound selected from the group consisting of:
methyl 2-(2-((t-butoxycarbonyl)amino)-3-hydroxypropanamido)-4-methylpentanoate;
methyl 2-(2-amino-3-hydroxypropanamido)-4-methylpentanoate;
methyl 2-(2-((t-butoxycarbonyl)amino)-4-methylpentanamido)acetate;
methyl 2-(2-amino-4-methylpentanamido)acetate;
methyl 2-(2-((t-butoxycarbonyl)amino)-3-methylbutanamido)acetate;
methyl 2-(2-amino-3-methylbutanamido)acetate;
methyl 2-(2-((t-butoxycarbonyl)amino)-3-hydroxybutanamido)-4-methylpentanoate;
methyl 2-(2-amino-3-hydroxybutanamido)-4-methylpentanoate;
ethyl 2-(2-((t-butoxycarbonyl)amino)-4-methylpentanamido)propanoate;
ethyl 2-(2-amino-4-methylpentanamido)propanoate;
2-(2-amino-4-methylpentanamido)propanoic acid;
methyl 2-(2-((t-butoxycarbonyl)amino)-3-methylbutanamido)propanoate;
methyl 2-(2-((t-butoxycarbonyl)amino)-propanamido)propanoate;
Methyl 2-(2-((t-butoxycarbonyl)amino)-ethanamido)propanoate;
methyl 2-(2-((t-butoxycarbonyl)amino)-3-((4-hydroxyphenyl)propanamido)propanoate;
methyl 2-(2-((t-butoxycarbonyl)amino)-acetic propanamido)-3-(indole-3yl)propanoate;
methyl 2-(2-((t-butoxycarbonyl)amino)-3-methylbutanamido)-3-(indole-3yl)propanoate;
methyl 1-(2-((t-butoxycarbonyl)amino)-3-phenylpropane oil)pyrolidine-2-carboxylate;
methyl 1-(2-((t-butoxycarbonyl)amino)propane oil)pyrolidine-2-carboxylate;
methyl 1-(2-((t-butoxycarbonyl)amino)-3-methylbutane oil)pyrolidine-2-carboxylate;
methyl 1-(2-((t-butoxycarbonyl)amino)-4-methylpentane oil)pyrolidine-2-carboxylate;
methyl 1-(5-amino-2-((t-butoxycarbonyl)amino)-5-oxopentane oil)pyrolidine-2-carboxylate;
methyl 1-(2-((t-butoxycarbonyl)amino)-3-hydroxybutane oil)pyrolidine-2-carboxylate;
methyl 1-(4-t-butoxy-2-((t-butoxycarbonyl)amino)-4-oxobutane oil)pyrolidine-2-carboxylate;
methyl 1-(4-amino-2-((t-butoxycarbonyl)amino)-4-oxobutane oil)pyrolidine-2-carboxylate;
t-butyl 2-((2-methoxycarbonyl)pyrolidine-1-carbonyl)pyrolidine-1-carboxylate;
methyl 1-((2,6-bis(t-butoxycarbonyl)amino)hexane oil)pyrolidine-2-carboxylate;
methyl 1-(2-((t-butoxycarbonyl)amino)-3-(imidazole-4-yl)propane oil)pyrolidine-2-carboxylate;
methyl 1-(5-t-butoxy-2-((t-butoxycarbonyl)amino)-5-oxopentane oil)pyrolidine-2-carboxylate;
methyl 1-(2-((t-butoxycarbonyl)amino)-3-(4-hydroxyphenyl)propanoyl)pyrolidine-2-carboxylate;
methyl 1-(2-((t-butoxycarbonyl)amino)-3-methylpentane oil)pyrolidine-2-carboxylate;
methyl 1-(2-((t-butoxycarbonyl)amino)acetyl)pyrolidine-2-carboxylate;
methyl 1-(2-((t-butoxycarbonyl)amino)-3-hydroxypropane oil)pyrolidine-2-carboxylate;
methyl 1-(2-((t-butoxycarbonyl)amino)-4-methylthiobutane oil)pyrolidine-2-carboxylate;
methyl 1-(2-((t-butoxycarbonyl)amino)-3-(indole-3-yl)propane oil)pyrolidine-2-carboxylate;
methyl 1-(2-((t-butoxycarbonyl)amino)-3-trithylthiopropane oil)pyrolidine-2-carboxylate;
methyl 1-(2-((t-butoxycarbonyl)amino)-3-mercaptopropane oil)pyrolidine-2-carboxylate;
methyl 2-(2-((t-butoxycarbonyl)amino)-4-methylpentanamido))-3-hydroxybutanoate;
ethyl 2-(2-((t-butoxycarbonyl)amino)-3-methylpentanamido))-propanoate;
methyl 2-(2-((t-butoxycarbonyl)amino)-3-hydroxypropanamido))-acetate;
ethyl 2-(2-((t-butoxycarbonyl)amino)-ethanamido))-propanoate;
methyl 2-(2-((t-butoxycarbonyl)amino)-4-methylpentanamido))-3-phenylpropanoate;
methyl 2-(2-((t-butoxycarbonyl)amino)-ethanamido)-3-methylbutanoate;
methyl 2-(2-amino-ethanamido)-3-methylbutanoate;
3-(1-(methoxycarbonyl)-2-phenylethylcarbamoyl)-3-aminopropanoic acid;
methyl 3-(1-methoxycarbonyl-2-phenylethylcarbamoyl)-3-(t-butoxycarbonylamino)propanoate;
methyl 3-(1-(methoxycarbonyl)-2-phenylethylcarbamoyl)-3-aminopropanoate;
allyl 3-(1-(methoxycarbonyl)-2-phenylethylcarbamoyl)-3-(N-butoxycarbonylamino)propanoate;
allyl 3-(1-(methoxycarbonyl)-2-phenylethylcarbamoyl)-3-aminopropanoate;
benzyl 3-(1-(methoxycarbonyl)-2-phenylethylcarbamoyl)-3-(N-butoxycarbonylamino)propanoate;
benzyl 3-(1-(methoxycarbonyl)-2-phenylethylcarbamoyl)-3-aminopropanoate;
methyl 3-(1-(methoxycarbonyl)-2-phenylethylcarbamoyl)-3-(N-acetylamino)propanoate;
methyl 3-(1-(methoxycarbonyl)-2-phenylethylcarbamoyl)-3-(N-benzoylamino)propanoate;
allyl 3-(1-(methoxycarbonyl)-2-phenylethylcarbamoyl)-3-(N-acetylamino)propanoate;
allyl 3-(1-(methoxycarbonyl)-2-phenylethylcarbamoyl)-3-(N-benzoylamino)propanoate;
benzyl 3-(1-(methoxycarbonyl)-2-phenylethylcarbamoyl)-3-(N-acetylamino)propanoate;
benzyl 3-(1-(methoxycarbonyl)-2-phenylethylcarbamoyl)-3-(N-benzoylamino)propanoate;
3-(1-(methoxycarbonyl)-2-phenylethylcarbamoyl)-3-(N-acetylamino)propanoic acid;
methyl 2-(2-acetylamino)-3-methylbutanamido)acetate;
methyl 2-(2-acetylamino)-4-methylpentanamido)acetate;
methyl 2-((2-pyrolidinecarbamoyl)amino)acetate;
methyl 1-(2-amino-3-(indole-3-yl)propanoyl)pyrolidine-2-carboxylate;
methyl 2-(2-amino-3-methylbutanamido)propanoate;
methyl 2-(2-amino-3-methylpentanamido)propanoate; and
the agro-pharmaceutically acceptable salts thereof.

5. The agricultural plant-protecting agent according to claim 1, the agricultural plant-protecting agent being formulated in various types, such as, water dispersible powders, suspension, emulsion, emulsifiable concentrate, microemulsion, liquid formulation, dispersible formulation, particulate water dispersible powders, granule, powdered medicine, liquid water dispersible powder, particulate water dispersible powder, water floating granule, or tablets.

6. The agricultural plant-protecting agent according to claim 1, the agricultural plant-protecting agent being have a plant disease-preventing effect, a plant growth-promoting effect, a plant immunity-activating effect, or the combined effect thereof.

7. The agricultural plant-protecting agent according to claim 6, wherein the plant disease is a bacterial rot, a damping-off, a phytophthora blight, a wilt disease, a leaf spot disease or a mosaic disease.

8. The agricultural plant-protecting agent according to claim 6, wherein the plant is dicotyledoneae.

9. The agricultural plant-protecting agent according to claim 8, wherein the dicotyledoneae is selected from the group consisting of potatoes, peppers, sweet peppers, tomatoes, cucumbers, cigarettes, watermelons, oriental melons, Chinese cabbage, lettuces, radishes, cabbages, rapes, peanuts, a salary, ginseng, dong quai, perilla seeds, strawberry, spring onion, garlic, ginger, onion, rice, barley, corn, sorghum, apple tree, pear tree, peach tree, and persimmon tree.

10. A method for promoting plant growth, the method comprising of treating the agricultural plant-protecting agent according to claim 1 to a plant.

11. The method according to claim 10, wherein the plant is dicotyledoneae.

12. The method according to claim 11, wherein the dicotyledoneae is selected from the group consisting of potatoes, peppers, sweet peppers, tomatoes, cucumbers, cigarettes, watermelons, oriental melons, Chinese cabbage, lettuces, radishes, cabbages, rapes, peanuts, a salary, ginseng, dong quai, perilla seeds, strawberry, spring onion, garlic, ginger, onion, rice, barley, corn, sorghum, apple tree, pear tree, peach tree, and persimmon tree.

13. A method for preventing or suppressing plant diseases, the method comprising of applying the agricultural plant-protecting agent according to claim 1 to a plant.

14. The method according to claim 13, wherein the plant is dicotyledoneae.

15. The method according to claim 14, wherein the dicotyledoneae is selected from the group consisting of potatoes, peppers, sweet peppers, tomatoes, cucumbers, cigarettes, watermelons, oriental melons, Chinese cabbage, lettuces, radishes, cabbages, rapes, peanuts, a salary, ginseng, dong quai, perilla seeds, strawberry, spring onion, garlic, ginger, onion, rice, barley, corn, sorghum, apple tree, pear tree, peach tree, and persimmon tree.

16. The method according to claim 13, wherein the plant disease is a bacterial rot, a damping-off, a phytophthora blight, a wilt disease, a leaf spot disease or a mosaic disease.

17. A method for activating plant immunity, the method comprising of treating the agricultural plant-protecting agent according to claim 1 to a plant.

18. The method according to claim 17, wherein the plant is dicotyledoneae.

19. The method according to claim 18, wherein the dicotyledoneae is selected from the group consisting of potatoes, peppers, sweet peppers, tomatoes, cucumbers, cigarettes, watermelons, oriental melons, Chinese cabbage, lettuces, radishes, cabbages, rapes, peanuts, a salary, ginseng, dong quai, perilla seeds, strawberry, spring onion, garlic, ginger, onion, rice, barley, corn, sorghum, apple tree, pear tree, peach tree, and persimmon tree.

20. The method according to claim 17, wherein a disease-resistant ability to a plant pathogen or plant cold weather damage-preventing ability is increased.
